(19) United States
(12) Patent Application Publication
(10) Pub. No.: US 2002/0044898 A1
(43) Pub. Date: Apr. 18, 2002

(54) Instrument securing device for a sterilizer

(75) Inventors: Roberto M. Sergio, Jenkintown, PA (US); Winfield Wood JR., Gywnedd, PA (US); Wesley Jue, Horsham, PA (US)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: SERMED INDUSTRIES, INC.

(21) Appl. No.: 09/998,589

(22) Filed: Nov. 16, 2001

Related U.S. Application Data
(63) Continuation-in-part of application No. 09/425,261, filed on Oct. 22, 1999.
(60) Provisional application No. 60/249,822, filed on Nov. 17, 2000.
(60) Provisional application No. 60/105,115, filed on Oct. 22, 1998.
(60) Provisional application No. 60/105,225, filed on Oct. 22, 1998.
(60) Provisional application No. 60/105,221, filed on Oct. 22, 1998.

(51) Int. Cl. A61L 2/16

(52) U.S. Cl. 422/300; 422/297

(57) ABSTRACT

An instrument holder for securing an instrument in a chamber of a sterilizer. The instrument has an external surface and a proximal end. The sterilizer has a source of fluid. The instrument holder comprises a housing, an instrument coupler and an instrument retaining assembly. The housing has a base with a bore therethrough. The instrument coupler is for engaging the proximal end of the instrument and is movable within the bore. The instrument retention assembly is connected to the base and comprises a first grip and a second grip for alternately securing the instrument to the base.

### CROSS-REFERENCE TO RELATED APPLICATIONS

This application is a continuation in part of U.S. patent application Ser. No. 09/425,261 entitled “Apparatus and Method for Sterilizing an Instrument at Substantially Room Temperature,” filed Oct. 22, 1999, which is hereby incorporated by reference herein in its entirety and additionally claims the benefit of Provisional Application No. 60/249,822 entitled “Instrument Securing Device for a Sterilizer,” filed Nov. 17, 2000, which is hereby incorporated by reference herein in its entirety.

U.S. patent application Ser. No. 09/425,261 claims priority from: U.S. Provisional Patent Application Ser. No. 60/105,115 entitled, “Method and Apparatus for the Sterilization of Dental Handpieces at Room Temperature” filed Oct. 22, 1998, which is hereby incorporated by reference herein in its entirety; U.S. Provisional Patent Application Ser. No. 60/105,225 entitled, “Apparatus for the Sterilization of Threaded Areas of Dental Handpieces” filed Oct. 22, 1998, which is hereby incorporated by reference herein in its entirety; and from U.S. Provisional Patent Application Ser. No. 60/105,221 entitled, “Cartridge Assembly for Sterilant Containment” filed Oct. 22, 1998, and is hereby incorporated by reference herein in its entirety.

### BACKGROUND OF THE INVENTION

The present invention relates to the sterilization of thermosensitive instruments and, more specifically, to a device for securing instruments in a sterilizer.

Current methods for sterilizing instruments include using steam autoclaves, using ethylene oxide, or using irradiation. While these methods are effective for sterilizing instruments, none of these methods are suitable for performing instrument sterilization at a patient side location while exposing the instrument to substantially room temperatures during the sterilization process.

Steam autoclaves operate at temperatures ranging between two hundred-forty degrees Fahrenheit and two hundred seventy-five degrees Fahrenheit for extended periods of time. The high temperatures used by steam autoclaves have been known to damage thermosensitive instruments, such as the turbines of a dental handpiece, and tend to reduce the useful life of the thermosensitive instruments. This results in the associated medical instruments requiring more frequent and expensive refurbishing.

Ethylene oxide is a carcinogenic, flammable, and highly toxic substance. Expensive ventilation systems are required before the discharge resulting from the ethylene oxide sterilization process is released to the atmosphere. Thus, the use of ethylene oxide raises safety issues with regard to the sterilization of instruments at a patient-side location. Problematic environmental issues are also associated with the use of ethylene oxide.

The use of irradiation for sterilization is not a practical solution for normal patient-side applications. Irradiation sterilization requires large and expensive installations and protective measures that make irradiation sterilization unsuitable for use at a patient-side location.

Currently, the pre-cleaning of soiled medical instruments prior to the exposure of the instrument to the actual sterilizing heat, chemicals, or radiation depends on manual cleaning which is performed by medical personnel. The reliance on medical personnel for the manual cleaning of instruments increases the chance of inadequate cleaning due to human error or due to the omission of pre-cleaning all together.

Chemical sterilization can be used to sterilize instruments at room temperature, but is difficult and hazardous to manually perform. One method of overcoming the difficulties of manual sterilization is to automate the process. However, one problem with automating a chemical sterilizing process is the need to have the sterilizing agent contact every surface of the instrument being sterilized. If the sterilizing agent does not contact all the exterior and interior surfaces of the instrument, the processed instrument will not necessarily be sterilized and can still be a host for various spores and pathogens.

What is needed, but so far not provided in the sterilizing art, is an instrument securing device for use in a sterilizer that allows a sterilizing agent to contact the entire outer surface of the medical instrument while maintaining the medical instrument in a secured position.

### BRIEF SUMMARY OF THE INVENTION

One aspect of the invention is an instrument holder for securing an instrument in a chamber of a sterilizer. The instrument has an external surface and a proximal end. The sterilizer has a source of fluid. The instrument holder comprises a housing, an instrument coupler and an instrument retention assembly. The housing has a base with a bore therethrough. The instrument coupler is for engaging the proximal end of the instrument and is movable within the bore. The instrument retention assembly is connected to the base and comprises a first grip and a second grip for alternately securing the instrument to the base.

Another aspect of the invention is an instrument holder for securing an instrument in a chamber of a sterilizer. The instrument has an external surface and a proximal end. The sterilizer has a source of fluid. The instrument holder comprises a housing, an instrument coupler and an instrument retention assembly. The housing has a base with a bore therethrough. The instrument coupler is for engaging the proximal end of the instrument and is movable within the bore. The instrument coupler comprises an instrument engaging end engagable with the proximal end of the instrument; an intake port for receiving fluid from the source of fluid; a passageway in fluid communication with the intake port; and a discharge port for discharging fluid to the exterior surface of the instrument. The discharge port is in fluid communication with the passageway when the proximal end of the instrument is engaged with the instrument engaging end of the coupler. The instrument retention assembly is connected to the base. The instrument retention assembly comprises a first grip comprising a first pair of opposing cams and a second grip comprising a second pair of opposing cams. Each cam is pivotably connected to the base and pivotable between a disengaged position and an engaged position.

Still another aspect of the invention is an instrument holder for securing an instrument in a chamber of a sterilizer. The instrument has an external surface, a proximal end and a plurality of lumens. The chamber has a sealable opening for receiving the instrument holder. The sterilizer has a source of fluid. The instrument holder comprises a housing, an instrument coupler and an instrument retention assembly. The housing has a base with a bore therethrough. The instrument coupler is for engaging the proximal end of the instrument. The instrument coupler is movable within the bore. The instrument coupler comprises an instrument engaging end engagable with the proximal end of the instrument, a position biasing member and an annular seal. The position biasing member is for maintaining a predetermined contact force between the instrument engaging end of the coupler and the proximal end of the instrument. The position biasing member has a first end connected to the housing and a second end connected to the coupler. The annular seal is in slideable frictional engagement with the bore. The instrument coupler additionally comprises an intake port, a manifold, a passageway, and a discharge port. The intake port is for receiving fluid from the source of fluid. The manifold is in fluid communication with the intake port. The passageway comprises a plurality of lumen engaging conduits. At least one conduit of the plurality of lumen engaging conduits is in fluid communication with the manifold. The discharge port is for discharging fluid to the exterior surface of the instrument. The discharge port is in fluid communication with the passageway when the proximal end of the instrument is engaged with the instrument engaging end of the instrument coupler. The instrument retention assembly is connected to the base. The instrument retention assembly comprises a first grip and a second grip. The first grip comprises a first pair of opposing cams comprising a first cam and a second cam. The first and second cams are driven by a first actuator and a second actuator, respectively. The first and second actuators are synchronized for common actuation. The second grip comprises a second pair of opposing cams comprising a third cam and a fourth cam. The third and fourth cams are driven by a third actuator and a fourth actuator, respectively. The third and fourth actuators are synchronized for common actuation. The first, second, third, and fourth cams are pivotably connected to the base and pivotable between a disengaged position and an engaged position. The first grip and the second grip form an arrangement operable in a plurality of configurations. In a disengaged configuration, each cam of the first and second grips is in the disengaged position and the proximal end of the instrument is removably engagable with the instrument coupler. In a first configuration, the first pair of opposing cams of the first grip is in the engaged position and secures the instrument to the base, and the exterior surface of the instrument along opposed first radial arcs is exposed. In a second configuration, the second pair of opposing cams of the second grip is in the engaged position and secures the instrument to the base, and the exterior surface of the instrument along opposed second radial arcs is exposed. In a third configuration, the first pair of cams and the second pair of cams are in the engaged position and secure the instrument to the base, and the exterior surface of the instrument along a plurality of third radial arcs is exposed.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

In the drawings:

FIG. 1 is a schematic of a sterilizer incorporating a preferred embodiment of an instrument holder according to the present invention;

FIG. 2 is an enlarged partial view of a proximal end of the instrument shown in FIG. 1;

FIG. 3 is an enlarged partial cross-sectional view of a portion of the instrument after the instrument has been securely mounted in an interior compartment of a chamber of FIG. 1;

FIG. 4 is a partial cross-sectional view of the instrument mounted within the interior compartment of the chamber of FIG. 1;

FIG. 5 is a top plan view of the instrument holder of FIG. 1;

FIG. 6 is a cross-sectional view of the instrument holder of FIG. 1, taken along the line 6-6 of FIG. 5;

FIG. 7 is a cross-sectional view of the instrument holder of FIG. 1, taken along the line 7-7 of FIG. 5;

FIG. 8 is a top schematic view of the pneumatic lines for actuating the actuators and the line supplying the source of fluid for the instrument holder of FIG. 1;

FIG. 9 is a schematic diagram illustrating the relative positions of the instrument, a retainer ring, and the first through fourth cams of the instrument holder of FIG. 5 when the first and second grips are in a disengaged configuration;

FIG. 10 is a schematic diagram illustrating the relative positions of the instrument, a retainer ring, and the first through fourth cams of the instrument holder of FIG. 5 when the first and second grips are in a first configuration;

FIG. 11 is a schematic diagram illustrating the relative positions of the instrument, a retainer ring, and the first through fourth cams of the instrument holder of FIG. 5 when the first and second grips are in a second configuration;

FIG. 12 is a schematic diagram illustrating the relative positions of the instrument, a retainer ring, and the first through fourth cams of the instrument holder of FIG. 5 when the first and second grips are in a third configuration;

FIG. 13 is a cross-sectional view of a nozzle assembly, which is not mounted into the chamber of FIG. 1;

FIG. 14 is an exploded perspective view of the nozzle assembly of FIG. 13;

FIG. 15 is a perspective view of the exploded nozzle assembly of FIG. 13 aligned for insertion into the chamber of FIG. 1; and

FIG. 16 is a cross-sectional view of a third preferred embodiment of the instrument retention assembly according to the present invention.

### DETAILED DESCRIPTION OF THE INVENTION

Certain terminology is used in the following description for convenience only and is not limiting. The words “right,” “left,” “lower” and “upper” designate directions in the drawings to which reference is made. The words “inwardly” and “outwardly” refer to directions toward and away from, respectively, the geometric center of the instrument holder and designated parts thereof. The terminology includes the words above specifically mentioned, derivatives thereof, and words of similar import.

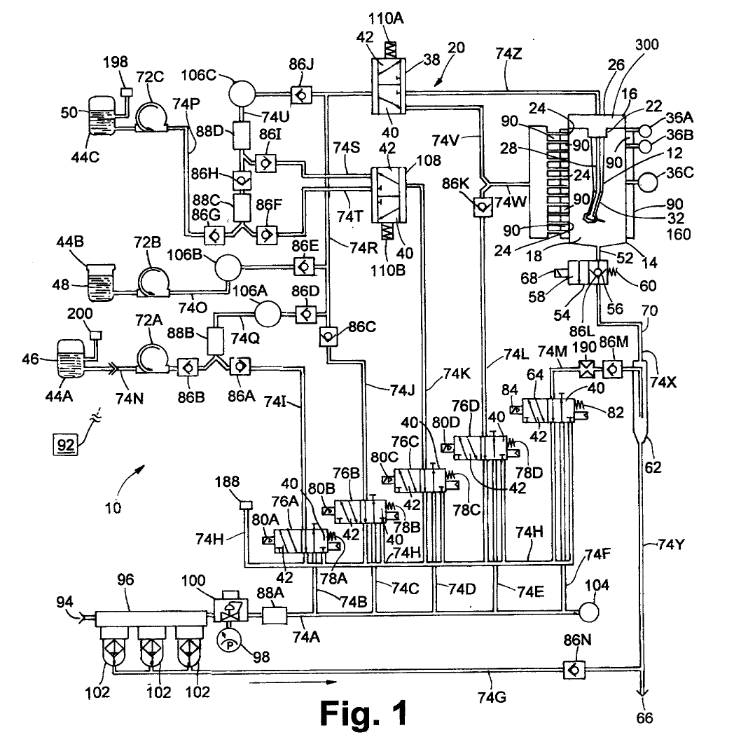

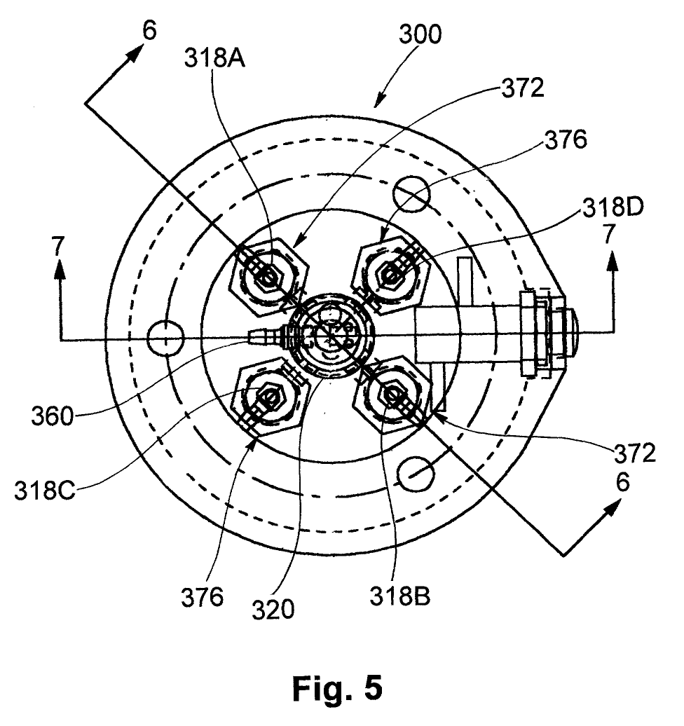

Referring to the drawings in detail, wherein like numerals indicate like elements throughout, there is shown in FIGS. 5-8 a first preferred embodiment of an instrument holder, generally designated 300 and hereinafter referred to as the “instrument holder” 300 in accordance with the present invention. The instrument holder 300 is for securing an instrument 12 in a chamber 14 of a sterilizer 10 (See FIG. 1).

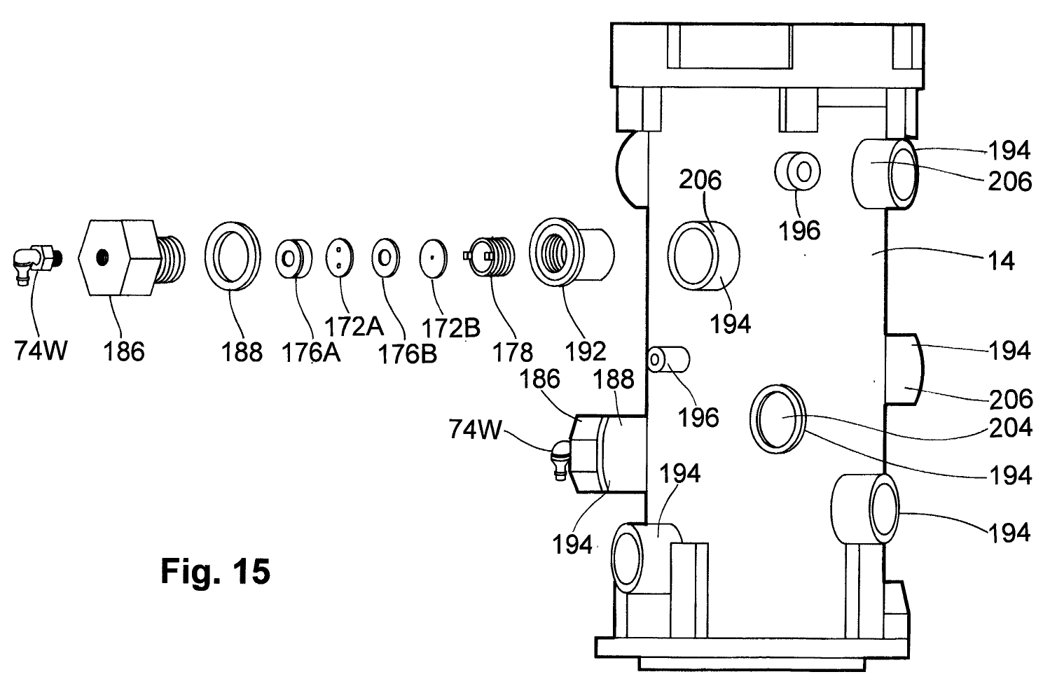

The preferred sterilizer 10 for use with the present invention is shown in FIG. 1 and is described in detail in U.S. patent application Ser. No. 09/425,261 which was filed on Oct. 22, 1999 and which is hereby incorporated by reference herein in its entirety. Referring to FIGS. 1, 3 and 4, the sterilizer 10 includes a chamber 14 having an interior compartment 18 for receiving and housing the instrument 12 and a source of fluid 46, 48, 50 for use in sterilizing the instrument 12. Referring to FIG. 15, the chamber 14 is preferably generally cylindrically shaped. However, those of skill in the art will appreciate from this disclosure that the instrument holder 300 of the present invention is not limited to use in any particular sterilizer or a sterlilzer having a chamber of any particular shape. For example, the chamber 14 may be rectangularly shaped, triangularly shaped, cubically shaped or the like without departing from the scope of the present invention.

The preferred instrument 12 for use with the present invention is a dental handpiece. However, those of skill in the art will appreciate from this disclosure that instruments other than dental handpieces can be sterilized using the instrument holder 300 of the present invention. For example, scalpels, forceps, prongs, tubes, trays, or any instrument used in a sterile lab, operating room, manufacturing site or the like can be sterilized in a quick and convenient manner using the apparatus 10 and the method of the present invention. Accordingly, while the preferred instrument 12 is discussed below as having an interior 28 (as is common in dental handpieces), those of skill in the art will appreciate from this disclosure that the present invention is not limited to instruments 12 having an interior 28. Thus, instruments such as a scalpel or the like can be used with the apparatus 10 or method of the present invention without departing from the scope of the invention. The necessary changes to the instrument holder 300 to accommodate instruments 12 other than the preferred dental handpiece would be obvious to one of ordinary skill in the art when considered in combination with this disclosure. Accordingly, for brevity, the below disclosure is directed to an instrument 12 having features that correspond to the general features of a dental handpiece, with the understanding that the invention is not limited to dental handpieces.

Referring to FIGS. 1-4 and 6-7, the preferred instrument 12 for use with the instrument holder 300 of the present invention is shown. The instrument 12 has an exterior surface 32 and a proximal end 162 that is attachable to the chamber 14. The distal end 164 of the instrument 12 houses a rotary turbine 160. The instrument 12 has a first lumen 128A for injecting fluid, preferably air, into the instrument 12 to turn the rotary turbine 160 and a second lumen 128B for discharging injected fluid from the instrument 12. The first and second lumens 128A, 128B extend from the proximal end 162 of the instrument 12. A third lumen 124A and a fourth lumen 124B transport air and water, respectively, to the distal end of the instrument 12. A portion of the proximal end 162 of the instrument 12 preferably has threads 132 for securing the instrument to the appropriate dental apparatus (not shown).

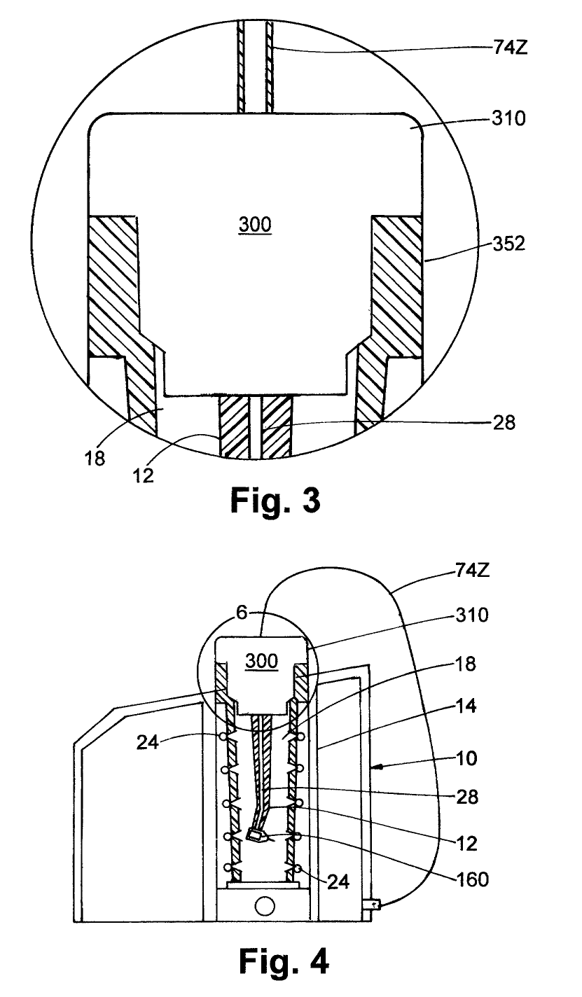

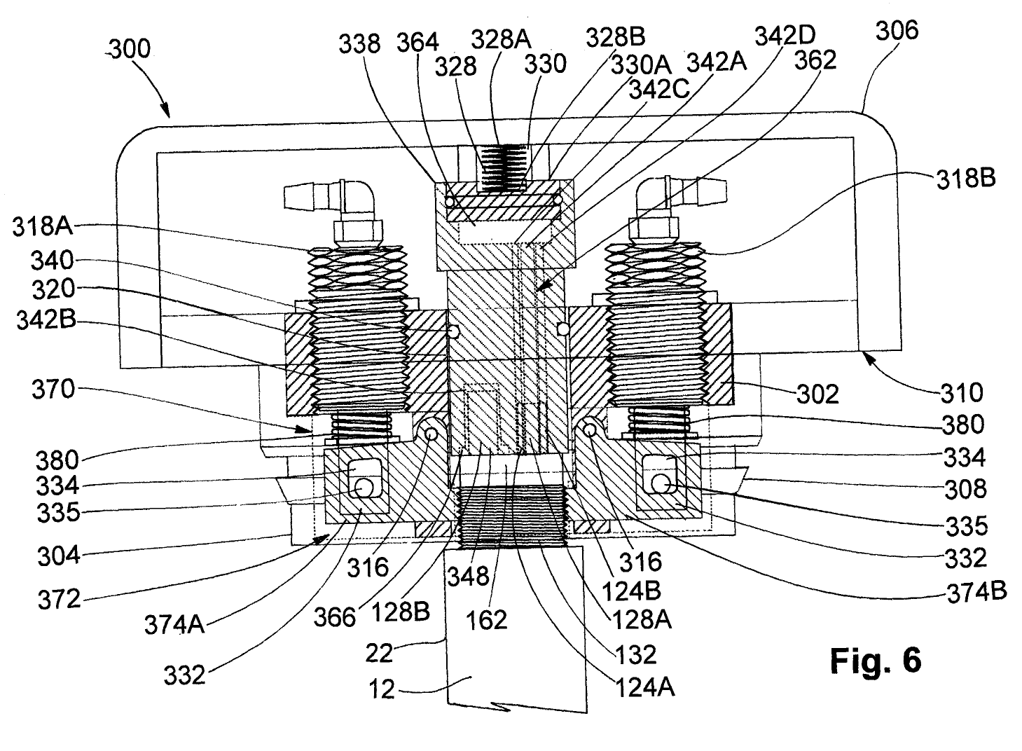

Referring to FIGS. 5-7, the instrument holder 300 preferably has a housing 310 comprising a base 302 supported by a mount 304 covered by a lid 306. The lid 306 preferably is detachably engaged to the mount 304 to allow easy access to lumen engaging conduits 342A, 342B, 342C, 342D (collectively referred to as the “lumen engaging conduits 342”) for easy maintenance and repair. The lumen engaging conduits 342 are further described below in reference to FIG. 6. The base 302 preferably is secured to the mount 304 in an interference fit. Those having ordinary skill in the art will understand from the present disclosure that the base 302 may be secured to the mount 304 by a variety of well known fastening methods such as screws or the like without departing from the spirit and scope of the invention. The mount 304 preferably has a seal 308 for sealing the chamber 14 when the mount 304 is detachably engaged to the chamber 14. Preferably, the chamber 14 has a collar 352 (FIG. 3) integral therewith. The collar 352 has a sealable opening having a shape corresponding to the shape of the mount 304 for receiving the mount 304.

Those of ordinary skill in the art will appreciate from this disclosure that various methods of positioning the instrument holder 300 within the chamber 14 can be used without departing from the present invention. For example, the collar 352 and the mount 304 can have a bayonet type interface, a threaded interface or the like without departing from the scope of the present invention. Further, the artisan will understand that the collar 352 can be omitted and the mount 304 directly coupled to the chamber 14.

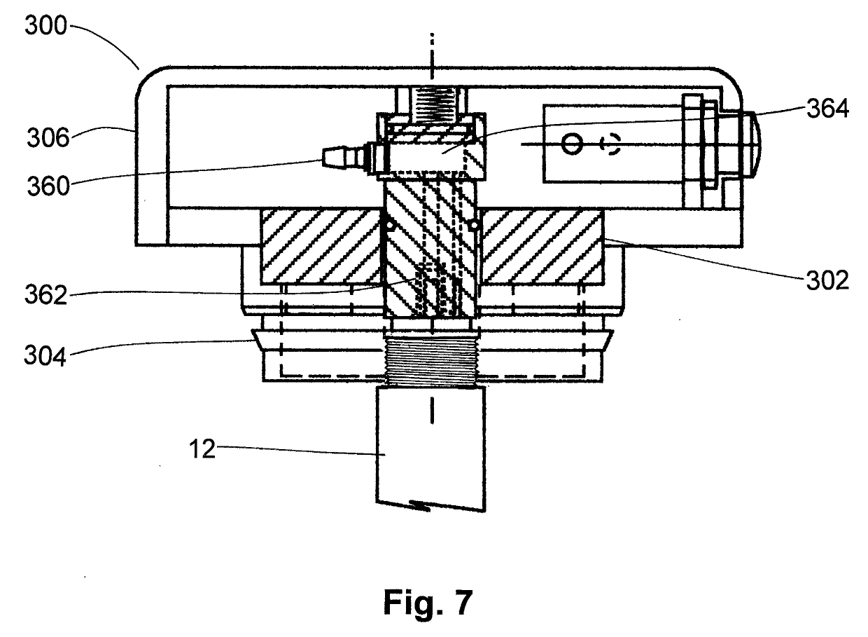

Referring to FIGS. 6-7, the base 302 has a bore 320 therethrough. The bore 320 is generally centrally positioned. Those of ordinary skill in the art will appreciate from this disclosure that the bore 320 can be positioned off center without departing from the scope of the present invention. The artisan will further understand that the bore 320 preferably is circular in cross-sectional shape. However, the bore 320 can have a cross-sectional shape that is rectangular, elliptical, elongated, triangular, irregular or the like to accommodate instruments 12 of various shapes.

The instrument holder 300 has an instrument coupler 338 that is movable within the bore 320. The instrument coupler 338 has an instrument engaging end 348 for engaging the proximal end 162 of the instrument 12. Preferably, the instrument coupler 338 has a position biasing member 328 for maintaining a predetermined contact force between the instrument engaging end 348 of the instrument coupler 338 and the proximal end 162 of the instrument 12. The position biasing member 328 has a first end 328A connected to the housing 310 and a second end 328B connected to the instrument coupler 338. The position biasing member 328 preferably is a compression spring that is retained within a channel 330 in the lid 306. The channel 330 has a first end 330A that serves as a stop limiting both the travel of the coupler 338 and the force applied by the position biasing member 328 to the coupler 338. Those having ordinary skill in the art will understand that the position biasing member is not restricted to a compression spring and can be any elastic material that applies to the coupler 338 a position biasing force without departing from the scope of the invention. The magnitude of the predetermined force is dependent on the pressure, preferably about six (6) pounds per square inch, of the fluids within the first through fourth lumens 128A, 128B, 124A, 124B during sterilization of the instrument 12. Preferably, the instrument coupler 338 has an annular seal 340 in slideable frictional engagement with the bore 320.

The instrument coupler 338 has an intake port 360 for receiving fluid from the source of fluid 46, 48, 50 and a passageway 362 in fluid communication with the intake port 360. The passageway 362 preferably has a plurality of lumen engaging conduits 342, and the instrument coupler 338 preferably further comprises a manifold 364 in fluid communication with the intake port 360 and with at least one conduit of the plurality of lumen engaging conduits 342. The plurality of lumen engaging conduits 342 preferably includes a first lumen engaging conduit 342A and a second lumen engaging conduit 342B. The first and second lumen engaging conduits 342A, 342B are positioned for engagement with the first and second lumens 128A, 128B, respectively, when the instrument engaging end 348 of the instrument coupler 338 is engaged with the proximal end 162 of the instrument 12.

The instrument coupler 338 additionally has a discharge port 366 for discharging fluid to the exterior surface 32 of the instrument 12. The discharge port 366 is in fluid communication with the passageway 362, and preferably with the second lumen engaging conduit 342B, when the proximal end 162 of the instrument 12 is engaged with the instrument engaging end 348 of the instrument coupler 338.

The instrument holder 300 has an instrument retention assembly 370 connected to the base 302. The first preferred embodiment of the instrument retention assembly 370 comprises a first grip 372 and a second grip 376 (FIG. 5) for alternately securing the instrument 12 to the base 302. The second grip 376 is substantially the same as the first grip 372. Accordingly, for brevity, only the features of the first grip 372 are shown in detail in FIG. 6. Those skilled in the art will understand from this disclosure that a side cross-sectional view of the instrument holder 300 perpendicular to the view in FIG. 6 would appear substantially the same as FIG. 6 and would show the second grip 376 having features corresponding to the features of the first grip 372 discussed below.

The first grip 372 preferably comprises a first pair of opposing cams comprising a first cam 374A and a second cam 374B. Each cam 374A, 374B is pivotably connected by a pivot pin 316 to the base 302 and pivotable between a disengaged position, in which each cam 374A, 374B does not apply a force to the instrument 12, and an engaged position, in which each cam 374A, 374B applies a force to the instrument 12, as further discussed below.

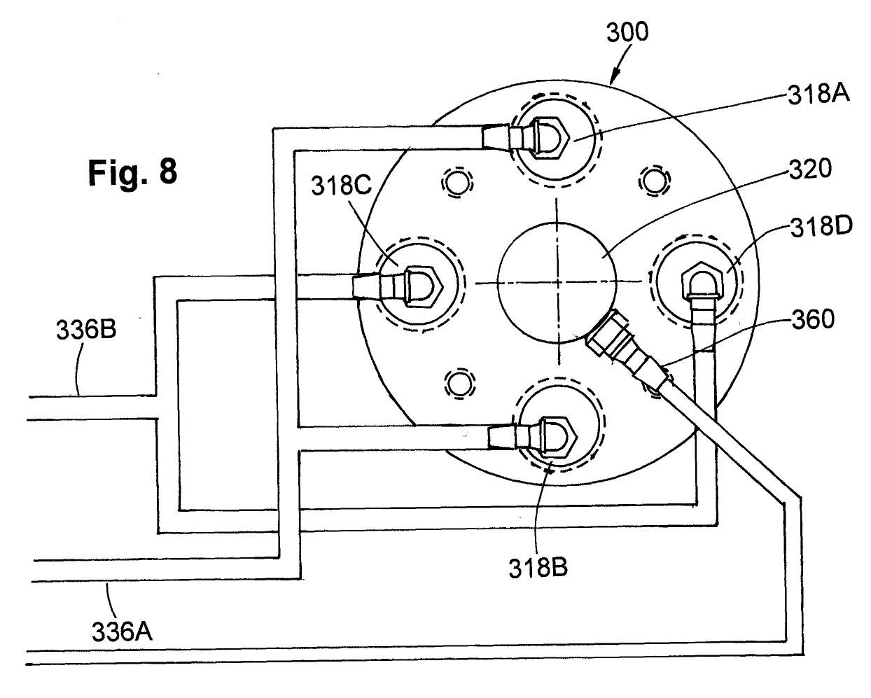

The first grip 372 preferably has a first actuator 318A and a second actuator 318B that drive the first and second cams 374A, 374B, respectively. The first and second actuators 318A, 318B are synchronized for common actuation. Preferably, the first and second actuators 318A, 318B are pneumatic actuators that can be activated by a common first pneumatic line 336A shown in FIG. 8. The first and second actuators 318A, 318B preferably have a reciprocable cam block 332. Preferably the first and second cams 374A, 374B are pivotably and slidably connected to the respective cam blocks 332 of the first and second actuators 318A, 318B by a slider pin 335 inserted in a slot 334 in the cam blocks 332. The first and second actuators 318A, 318B preferably are configured with actuator springs 380 to bias the first and second cams 374A, 374B in the disengaged position.

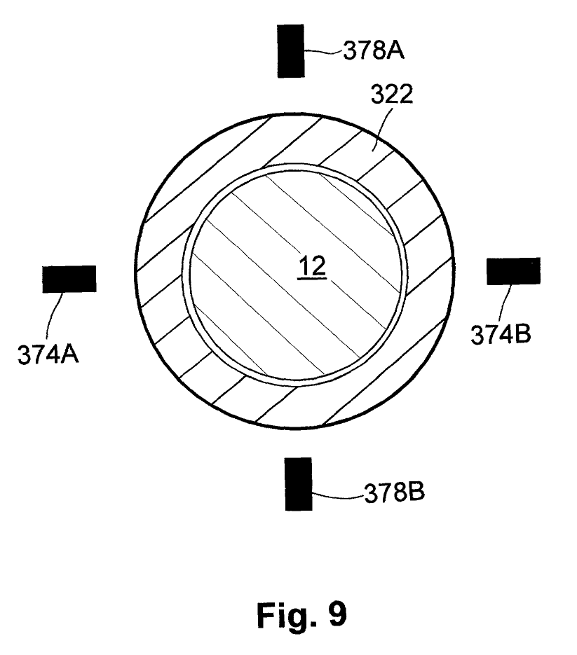

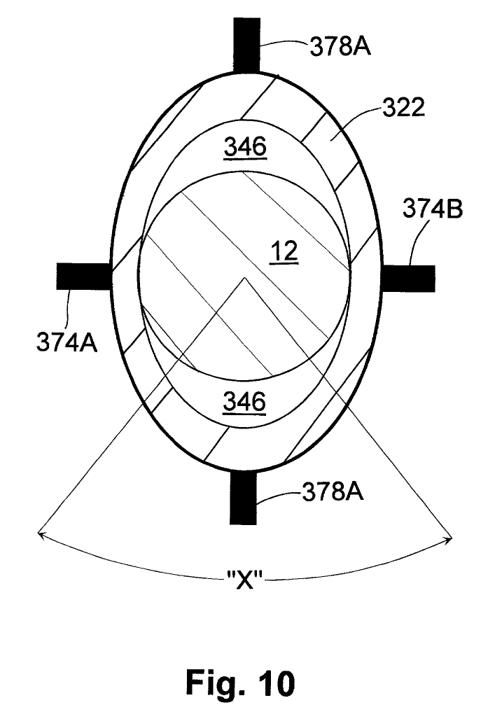

Referring to FIGS. 5, 8 and the above discussion regarding the first grip 372, the second grip 376 preferably comprises a second pair of opposing cams 378A, 378B comprising a third cam 378A and a fourth cam 378B. The third and fourth cams 378A, 378B are shown schematically in FIGS. 9-12 further discussed below. Each cam 378A, 378B is pivotably connected to the base 302 and pivotable between a disengaged position, in which each cam 378A, 378B does not apply a force to the instrument 12, and an engaged position, in which each cam 378A, 378B applies a force to the instrument 12, as further discussed below.

The second grip 376 preferably has a third actuator 318C and a fourth actuator 318D that drive the third and fourth cams 378A, 378B, respectively. The third and fourth actuators 318C, 318D are synchronized for common actuation. Preferably, the third and fourth actuators 318C, 318D are pneumatic actuators that can be activated by a common second pneumatic line 336B, as shown in FIG. 8. The third and fourth actuators 318C, 318D preferably are configured with actuator springs (not shown) to bias the third and fourth cams 378A, 378B in the disengaged position.

Referring to FIGS. 5 and 8, preferably the first, second, third, and fourth actuators 318A, 318B, 318C, 318D are equidistantly spaced around the bore 320 and are threadedly attached to base 302.

In a second preferred embodiment of the instrument retention assembly 370, a retainer ring 322, schematically shown in FIGS. 9-12, is positioned between the first grip 372 and the instrument 12 and between the second grip 376 and the instrument 12. Those having ordinary skill in the art will appreciate from the present disclosure that a portion of the mount 304 that extends below the first and second grips 372, 276 can be readily adapted to support the retaining ring 322 in the instrument holder 300 when neither the first grip 372 nor the second grip 376 is engaged with the retainer ring 322. The retainer ring 322 is preferably formed from a resilient material that allows the first and second grips 372, 376 to grasp the instrument 12 without damaging the threads 132, of the exterior surface 32 of the instrument 12.

The first grip 372 and the second grip 376 of the first and second preferred embodiments form an arrangement that is operable in a plurality of configurations. Since the plurality of configurations for the arrangement of the first and second grips 372, 376 are substantially the same with and without the presence of the retainer ring 322, for brevity, the configurations will be discussed with the retainer ring 322 present. However, those having ordinary skill in the art will understand from this disclosure that the retainer rings 322 can be omitted without departing from the scope of the present invention.

Referring to FIG. 9, when the first grip 372 and the second grip 376 are in a disengaged configuration, each cam 374A, 374B, 378A, 378B of the first and second grips 372, 376 is in the disengaged position. The retainer ring 332 is spaced from both the first and second grips 372, 376 and the instrument 12. In the disengaged configuration, the proximal end 162 of the instrument 12 is removably engagable with the instrument coupler 338 and inside the retainer ring 332.

Referring to FIG. 10, when the first grip 372 and the second grip 376 are in a first configuration, the first pair of opposing cams 374A, 374B of the first grip 372 is in the engaged position and secures the instrument 12 to the base 302 by applying a force to the retainer ring 322 which in turn engages a portion of the exterior surface 32 of the instrument. In the first configuration, the exterior surface 32 of the instrument 12 along opposed first radial arcs “X” is exposed and opposed first gaps 346A are formed between the exterior surface 32 of the instrument 12 and the retainer ring 322.

The formation of the first gaps 346A preferably causes portions of the exterior surface of the instrument 12, each along one of the opposed first radial arcs “X,” to be exposed to whatever fluid is currently being used with the instrument 12. It is preferable, but not necessary, that the first radial arcs be approximately one hundred seven (107°) degrees. Those of ordinary skill in the art will appreciate from this disclosure that the first radial arcs can have a range of between about one hundred eighty (180°) degrees and about ninety (90°) degrees without departing from the scope of the present invention

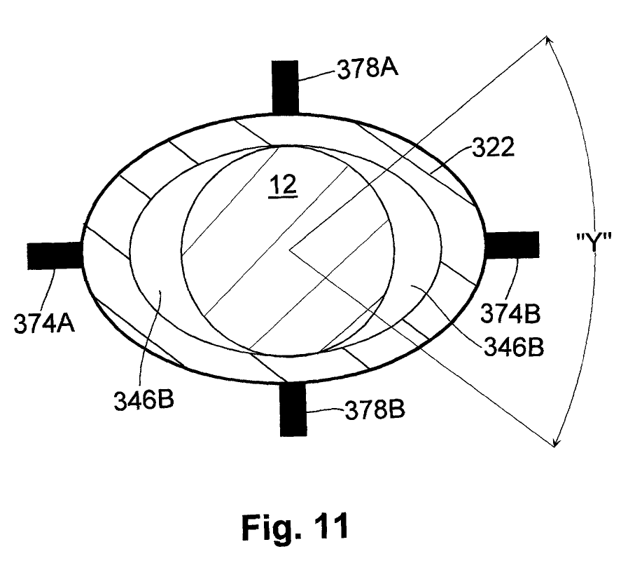

Referring to FIG. 11, when the first grip 372 and the second grip 376 are in a second configuration, the second pair of opposing cams 378A, 378B of the second grip 376 is in the engaged position and secures the instrument 12 to the base 302 by applying a force to the retainer ring 322 which in turn engages a portion of the exterior surface 32 of the instrument. In the second configuration, the exterior surface 32 of the instrument 12 along opposed second radial arcs “Y” is exposed and opposed second gaps 346B are formed between the exterior surface 32 of the instrument 12 and the retainer ring 322.

The formation of the second gaps 346B preferably causes portions of the exterior surface of the instrument 12, each along one of the second radial arcs “Y,” to be exposed to whatever fluid is currently being used with the instrument 12. It is preferable, but not necessary, that the second radial arcs be approximately one hundred seven (107°) degrees. Those of ordinary skill in the art will appreciate from this disclosure that the second radial arcs can have a range of between about one hundred eighty (180°) degrees and about ninety (90°) degrees without departing from the scope of the present invention. Additionally, the second radial arcs “Y” can be different from the first radial arcs “X” without departing from the scope of the present invention.

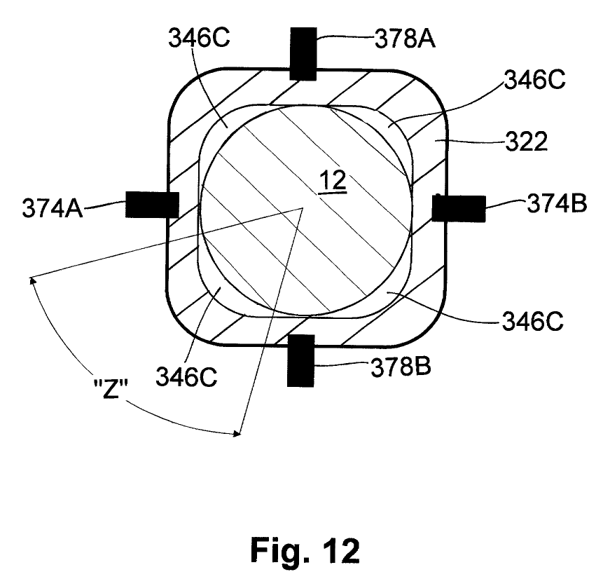

Referring to FIG. 12, when the first grip 372 and the second grip 376 are in a third configuration, the first pair of cams 374a, 374B and the second pair of cams 378A, 378B are in the engaged position and secure the instrument 12 to the base 302 by applying a force to the retainer ring 322 which in turn engages a portion of the exterior surface 32 of the instrument. In the third configuration, the exterior surface 32 of the instrument 12 along a plurality of third radial arcs “Z” is exposed and opposed third gaps 346C are formed between the exterior surface 32 of the instrument 12 and the retainer ring 322.

The formation of the third gaps 346C preferably causes portions of the exterior surface of the instrument 12, each along one of the third radial arcs “Z,” to be exposed to whatever fluid is currently being used with the instrument 12. It is preferred, but not necessary, that the third radial arcs “Z” be approximately seventy-three (73°) degrees.

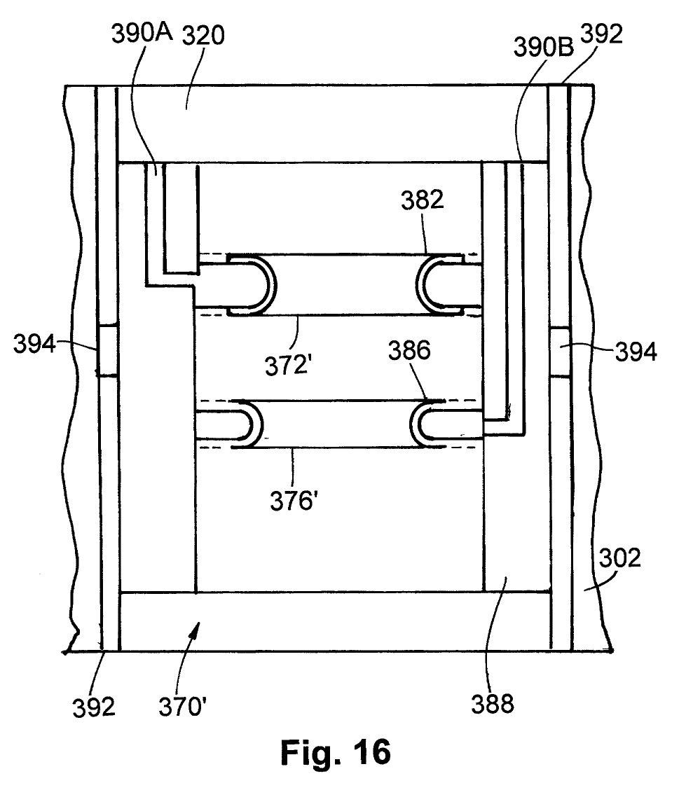

Referring to FIG. 16, there is shown a third preferred embodiment of the instrument retention assembly 370′. The instrument retention assembly 370′ comprises a first grip 372′ comprising a first ring 382 and a second grip 376′ comprising a second ring 386. The first and second rings 382, 386 are spaced apart and each has a radially inwardly facing surface that is pneumatically expandable. Preferably, the radially inwardly facing surface of the first and second rings are an elastomeric material. Preferably, the first and second rings 382, 386 are mounted, preferably with an interference fit, within a tubular core 388 that is removably insertable in the bore 320 of the base 302. Preferably, the tubular core 388 is fabricated from a polymeric material. Preferably, the tubular core 388 has a first channel 390A and a second channel 390B. The first channel 390A is in fluid communication with the source of pneumatic fluid 94 and additionally in fluid communication with the first ring 382. The second channel 390B is in fluid communication with the source of fluid 94 and additionally in fluid communication with the second ring 386.

Those having ordinary skill in the art will understand from this disclosure that the first and second rings 382, 386 must be sized to accommodate the proximal end 162 of the instrument 12 and have a radially inwardly facing surface having a first diameter that spaces the inwardly facing surface from the external surface 32 of the instrument 12 when the ring 382, 386 is deflated and a second diameter that allows the radially inwardly facing surface to secure the instrument 12 in the base 302 when the ring 382, 386 is inflated. Additionally, the artisan will understand that the tubular core 388 can be removably inserted and retained in the bore 320 of the base 302 in various well know ways, such as slots 392 in the bore 320 into which corresponding keys 394 integral with the tubular core 388 are insertable.

Referring to FIGS. 1, 8 and 9-12, during operation of the sterilizer 10 with the instrument holder 300 of the present invention, the first and second pneumatic lines 336A, 336B of the instrument holder 300 are connected to the source of pneumatic fluid 94, preferably air, under the control of a processor 92 controlling the sterilization of the instrument 12. Additionally, the intake port 360 is connected to the source of fluid 46, 48, 50. The instrument holder 300 preferably uses first grip 372 and the second grip 376 to alternately secure the instrument 12 in the instrument holder 300.

As best shown in FIG. 9, the instrument 12 is inserted in the instrument holder 300 while the first and second grips 372, 276 are arranged in the disengaged configuration. In the disengaged configuration, the first and second cams 374A, 374B and the third and fourth cams 378A, 378B and the retainer ring 322, if there is one, provide a clearance for the insertion of the proximal end 162 of the instrument 12 into the instrument holder 12. The instrument 12 is inserted in the instrument holder 12 until the instrument engaging end 348 engages the proximal end 162 of the instrument 12 and the position biasing member 328 applies the predetermined contact force between the instrument engaging end 348 of the instrument coupler 338 and the proximal end 162 of the instrument 12.

Referring to FIGS. 1, 8 and 10, to initially secure the instrument 12 in the instrument holder 300, the first and second grips 372, 276 are arranged in the first configuration. In the first configuration, the first and second actuators 318A, 318B are in fluid communication with the source of pneumatic fluid 94 via the first pneumatic conduit 336A to actuate the first grip 372, thereby securing the proximal end 162 of the instrument 12 between the first and second cams 374A, 374B of the first grip 372. The second grip 376 remains in the disengaged position. The instrument holder 300 with the instrument 12 secured thereto is inserted into the chamber 14.

The following description of the operation of the instrument holder 300 is presented with reference to a “predetermined step” of the sterilization process. The term “predetermined step” can be any step in the sterilization process and is used instead of specific reference to a particular sterilization step to emphasize that the preferred operation of the first and second grips 372, 376 can be used with any step in the sterilization process.

Referring to FIGS. 1, 8 and 11, once a portion of the predetermined step in the sterilizing process has been preformed while the instrument 12 is secured in the instrument holder 300 by the first grip 372, the first and second grips 372, 376 are arranged in the third configuration. In the third configuration, first and second actuators 318A, 318B remain in fluid communication with the source of pneumatic fluid 94. Additionally, the third and fourth actuators 318C, 318D are in fluid communication with the source of pneumatic fluid 94 via the second pneumatic conduit 366B, thereby also securing the proximal end 162 of the instrument 12 between the third and fourth cams 378A, 378B of the second grip 376.

Referring to FIGS. 1, 8 and 12, once the instrument 12 has been secured by both the first and second grips 372, 376, the first and second grips 372, 376 are arranged in the second configuration. In the second configuration, the first grip 372 the first and second actuators 318A, 318B are no longer in fluid communication with the source of pneumatic fluid 94 and return to the disengaged position. The third and fourth actuators 318C, 318D remain in fluid communication with the source of pneumatic fluid 94 to maintain actuation of the second grip 376, thereby securing the instrument 12 in the instrument holder 300 between the third and fourth cams 378A, 378B.

The preferred flow path for fluid from the lumen engaging conduits 342 to the first gaps 346A, as well as to the second and third gaps 346B, 346C, during the predetermined sterilization step is as follows. Fluid flows into the first lumen 128A and, in the preferred embodiment, is conveyed through the handpiece turbine 160. The fluid that enters the instrument 12 through the first lumen 128A is then exhausted through the second lumen 128B. The fluid that is exhausted through the second lumen 128B flows through a discharge port 366 between the instrument 12 and the coupling member 338. The expelled fluid then flows along the end of the instrument 12 and engages any threads 132 or portions of exterior surface 32 of the instrument 12 that are exposed via the first, second and third gaps 346A, 346B, 346C between the retainer ring 322 and the instrument 12. Fluid also flows into each of the third and fourth lumens 124A, 124B that are used to covey air and water, respectively, during the normal operation of the instrument 12.

Thus, by alternating the positions of the first, second and third gaps 346A, 346B, 346C around the proximal end 162 of the instrument 12, the entire exterior surface 32 of the instrument 12 can be exposed to any fluids, gases, or vapors used during the sterilization process. Additionally, due to the lumen engaging conduits 342 and the coupling member 338, any interior passageways, or surfaces, of the instrument are also exposed to any fluids, gases or vapors used during the sterilization process. As detailed below, the preferred embodiment of the present invention uses, but is not limited to, air, peracetic acid, sterilized water, and protease fluid.

The conduits used with the apparatus 10 are preferably polyethylene and/or nylon and have an external diameter of about four millimeters and an internal diameter of about two and one half millimeters. However, those of ordinary skill in the art will appreciate from this disclosure that the particular materials and size of the conduits can be changed without departing from the scope of the present invention. For example, any type of conduits can be used that can withstand the pressures, temperatures, and fluids used with the apparatus 10 without departing from the scope of the present invention. Additionally, the size of the conduits can be adjusted depending on the flow rates and pressures which are used with the apparatus 10 without departing from the scope of the present invention.

The chamber 14 is preferably formed of polyethylene tetrachloride. However, those of skill in the art will appreciate that the chamber 14 may be formed of any material having suitably low absorption and high acid resistance such as, inconnel, stainless steel, composites, or the like.

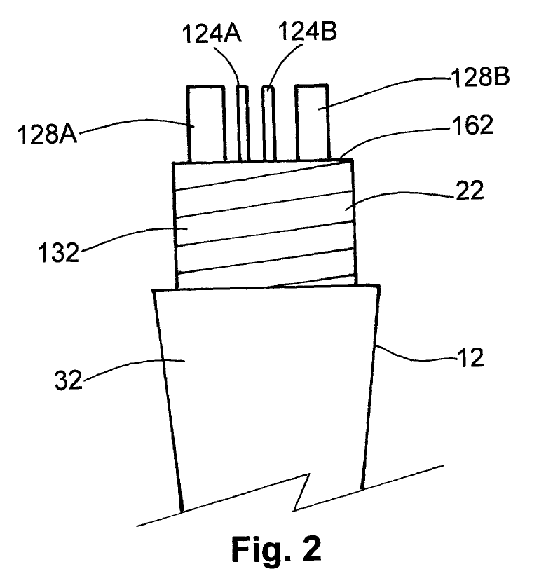

The interior compartment 18 is preferably maintained at a predetermined compartment temperature while the instrument 12 is being sterilized. The chamber 14 is releasably engagable with a portion 22 of the instrument 12 to support the instrument 12 within the interior compartment 18. Referring to FIGS. 2, 6, and 7, the portion 22 of the instrument 12 bears threads 132 which are used to attach the instrument 12 to an appropriate dental apparatus (not shown). Referring to FIGS. 3, 4, 6 and 7, the instrument 12 is attached to the instrument holder 300 by inserting the instrument 12 into the instrument holder 300.

Referring to FIG. 1, the chamber 14 preferably includes a first sensor 36A for detecting when the chamber 14 is closed. The first sensor 36A is preferably a non-contact magnetic proximity sensor of the well known in the art. However, those of skill in the art will appreciate from this disclosure that any sensor capable of determining when the lid 16 is secured to the chamber 14 can be used without departing from the scope of the present invention. A second sensor 36B detects when the instrument 12 is positioned within the interior compartment 18. The second sensor 36B is preferably an infrared sensor. However, those of skill in the art will appreciate from this disclosure that any sensor capable of detecting when the instrument 12 is positioned within the interior compartment 18 without interfering with the sterilization process can be used. A third sensor 36C detects a temperature of the interior compartment 18. The third sensor is preferably a thermocouple. However, those of ordinary skill in the art will appreciate from this disclosure that any sensor capable of detecting the compartment temperature can be used without departing from the present invention.

A controller 92 is operatively engaged with the chamber 14, a fluid injection mechanism 20 (further detailed below), the instrument retention assembly 370, the first sensor 36A, the second sensor 36B, and the third sensor 36C for regulating the flow of the fluid through the sterilizer 10 and the configuration in which the first and second grips 372, 376 are arranged. The controller 92 preferably uses an ATMEL 89C52 processor. However, those of ordinary skill in the art will appreciate from this disclosure that any suitable imbedded microprocessor assembly can be used to control and monitor the apparatus 10 without departing from the scope of the present invention. The processor is preferably attached to a customized control board having customized hardware interface electronics that are adapted for use with the sterilizing apparatus 10 and the instrument holder 300. Those of ordinary skill in the art will appreciate from this invention that a separate controller (not shown) can be disposed in the instrument holder 300 to operate the instrument holder 300.

A specially designed software program activates all the processes and monitors, in real time, the accuracy of the steps used to sterilize the instrument 12. A liquid crystal display (not shown) is preferably used to monitor the functions of the apparatus 10 while a printer (not shown) preferably prints out an operational log 134 detailing the various operations of the apparatus 10.

Referring to FIG. 15, the chamber 14 preferably has multiple nozzle receivers 194. Each nozzle receiver 194 preferably includes a tubular projection 206 which extends outwardly from the outer surface of the chamber 14. The tubular projections 206 enclose a chute 204 that extends through the tubular projection 206 and through the wall of the chamber 14. The chute 204 allows a nozzle (further detailed below) 24 to be secured therein. Two sensor receivers 196 are shown on the chamber 14. The sensor receiver 196 closer to the top of the chamber is preferably designed for use with the second sensor 36B which is used to determine whether an instrument 12 is positioned within the chamber 14. The sensor receiver 196 that is positioned closer to the bottom of the chamber 14 is preferably designed for use with the third sensor 36C which detects the temperature of the interior compartment 18.

While a preferred embodiment of the chamber has been described in detail above, those of skill in the art will appreciate from this disclosure that various structural features of the chamber 14 can be altered without departing from the scope of the present invention. For example, the particular connections between the instrument 12 and the chamber 14 may be varied as long as proper sterilization of the instrument 12 is not affected.

The fluid injection mechanism 20 is in fluid communication with the chamber 14 for supplying fluid to the chamber 14 and for maintaining the fluid at a predetermined fluid temperature while the instrument 12 is being sterilized. The fluid injection mechanism 20 uses a combination of fluid pumps (further detailed below) 72A-72C and pressurized air to transport appropriate fluids, further detailed below, through the chamber 14 for the cleaning and sterilizing of the instrument 12. The fluid is delivered by the fluid injection mechanism 20 to the chamber using either the twenty-sixth conduit 74Z or using a twenty-third conduit 74W.

The predetermined compartment temperature and the predetermined fluid temperature are preferably maintained within the range of between about fifty-five degrees Fahrenheit and about ninety-five degrees Fahrenheit during the sterilization of the instrument 12. This allows the instrument 12 to be sterilized while only being exposed to substantially room temperatures and thus prevents damage to thermosensitive instruments 12, such as dental handpieces. The currently preferred predetermined compartment temperature and the currently preferred predetermined fluid temperature are within the range of between about ninety degrees Fahrenheit and about ninety-four degrees Fahrenheit during the sterilization of the instrument 12. While preferred ranges have been detailed above, those of skill in the art will appreciate from this disclosure that the preferred temperature ranges assume an exposure of the instrument 12 to a sterilizing fluid 50 comprising a peracetic acid, further detailed below, for a time period between about three minutes and about six minutes. Additionally, the above temperature ranges are preferred for an apparatus 10 that completes the sterilization process, further detailed below, within a time period between of about ten minutes and about twelve minutes. Those of skill in the art will appreciate from this disclosure that if the time periods for completion of the sterilization process, or the associated exposure of the instrument to the sterilizing liquid were increased, or if a different type of sterilizing fluid were used with the apparatus then temperatures other than those detailed above could be used in combination with the apparatus 10 without departing from the scope of the present invention.

The fluid used by the apparatus 10 is any one of a rinse fluid 46, a bio-burden removal fluid 48, a sterilizing fluid 50, and filtered air. The rinse fluid 46 preferably comprises sterilized water. However, those of skill in the art will appreciate from this disclosure that any suitably sterile fluid capable of rinsing the instrument 12, which is safe for exposure to and consumption by patients can be used as the rinse fluid 46. The bio-burden removal fluid 48 preferably comprises a protease fluid. However, those of skill in the art will appreciate from this disclosure that any fluid capable of safely removing bio-burden from a soiled instrument 12 to simplify the killing of pathogen can be used as the bio-burden removal fluid 48. The sterilizing fluid 50 preferably comprises a peracetic acid. However, those of skill in the art will appreciate that the sterilizing fluid 50 may contain any components, which contribute to the killing of pathogens and are safe for use at a patient-side location.

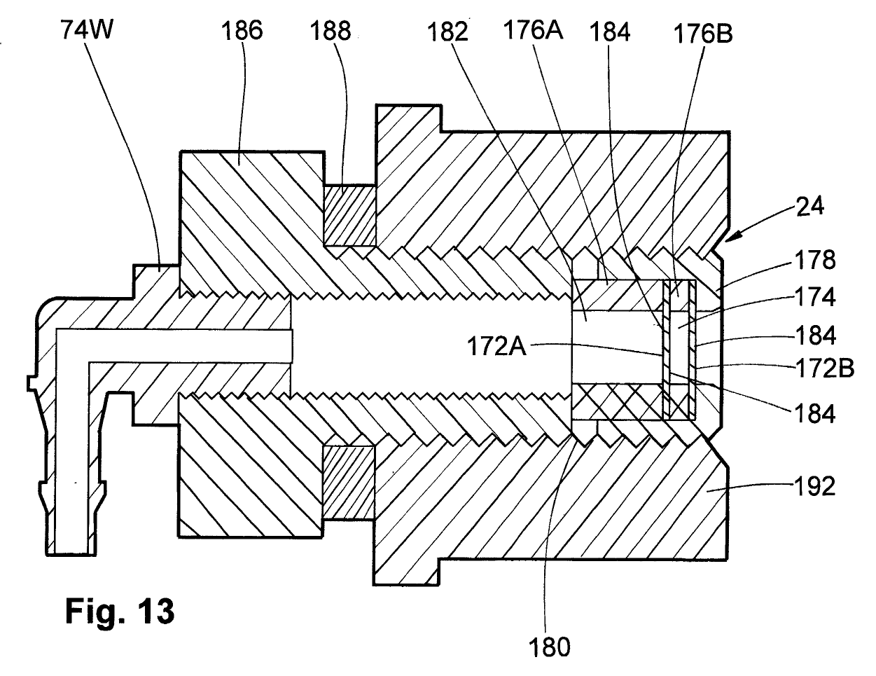

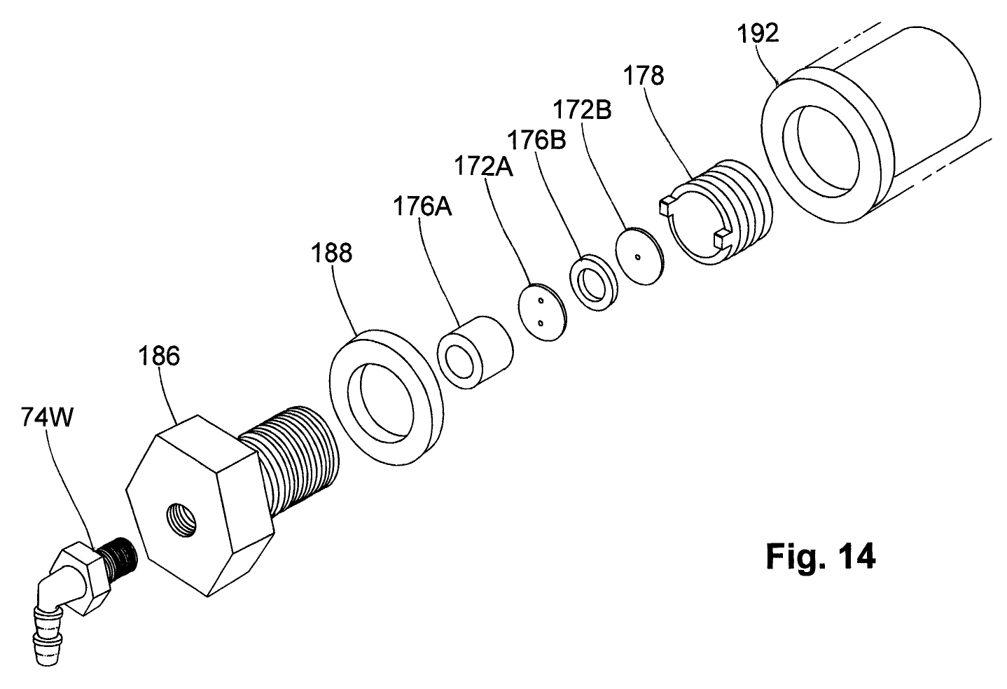

The chamber 14 includes at least one fluid outlet 24 for directing a flow of the fluid onto the exterior surface 32 of the instrument 12. Referring to FIGS. 1 and 13, twelve spaced fluid outlets 24 are preferably used in the chamber 14. However, those of skill in the art will appreciate from this disclosure that any number of fluid outlets 24 may be used to direct fluid onto the exterior surface 32 of the instrument 12 as long as proper amounts of the fluid can be directed onto the exterior surface 32 of the instrument 12. When the fluid injection mechanism 20 sends fluid to the fluid outlets 24, fluid is transported along the twenty-second conduit 74V to a twenty-third conduit 74W which guides the fluid into each of the fluid outlets 24.

The at least one fluid outlet 24 preferably, but not necessarily, comprises at least one nozzle 24 mounted to the chamber 14 to direct the flow of the fluid onto the exterior surface 32 of the instrument 12. Referring to FIGS. 13-15, each nozzle 24 is preferably inserted in the inner surface of the chamber 14. The spraying action of the nozzle is preferably caused by first and second nozzle plates 172A, 172B. The nozzle plates 172A, 172B are preferably disposed in a spaced apart parallel planar fashion to create a compartment 174 therebetween where turbulent fluid flow takes place as further detailed below. The nozzle 24 preferably includes a first and second generally annular spacer 176A, 176B, which are used to hold the first and second nozzle plates 172A, 172B in a spaced apart generally parallel planar fashion.

The first and second spacers 176A, 176B are preferably formed of a low absorption and non-reactive material such as nylon or the like. The first spacer 176A receives a fluid from the fluid injection mechanism 20, further detailed below. The first nozzle plate 172A has a first and second surface. The first surface is disposed on a distal end of the first spacer 176A and has at least one, but preferably two holes 184 extending therethrough. The second spacer 176B is disposed on the second surface of the first nozzle plate 172A and the second nozzle plate 172B is attached on an opposite end of the second spacer 176B from the first nozzle plate 172A and has a hole 184 therein. The first and second nozzle plates 172A, 172B and the first and second spacers 176A, 176B are held in position by a nozzle retainer 178. The nozzle retainer 178 is preferably circularly shaped to facilitate the threaded engagement between the nozzle retainer 178 and the threaded bore 180 which is disposed in a nozzle insert 192. It is preferable that the first and second nylon spacers 176A, 176B and the first and second nozzle plates 172A, 172B are circularly shaped. However, those of skill in the art will appreciate from this disclosure that the first and second nylon spacers 176A, 176B and the first and second nozzle plates 172A, 172B may have other shapes when viewed along the longitudinal axis of the nozzle 24 without departing from the scope of the present invention.

Referring to FIGS. 13-15, the first nozzle plate 172A is positioned on the right side of the first nylon spacer 176A to form a chamber 182. The second nylon spacer 176B is positioned on the opposite side of the first nozzle plate 172A from the first nylon spacer 176A. The second nozzle plate 172B is positioned on the right side of the second nylon spacer 176B to form the compartment 174.

The first nozzle plate 172A has two holes 184 which allow fluid to pass from the chamber 182 into the compartment 174. A single hole 184 is preferably positioned in the second nozzle plate 172B. The combination of the positioning of the two holes 184 in the first nozzle plate 172A and the positioning of the one hole 184 in the second nozzle plate 172B combine to generate a turbulent fluid flow within the compartment 174 which results in the emission of a vigorous spray of the fluid from the hole 184 in the second nozzle plate 172B. The turbulent fluid flow in compartment 174 results in the spray having a shape similar to a cone with an angular width of about ninety degrees as measured from the hole 184 in the second nozzle plate 172B.

The first and second nozzle plates 172A, 172B and the nozzle retainer 178 are preferably formed of inconnel but may be formed of any low absorption corrosion resistant material capable of withstanding the fluid pressures used by the apparatus 10 such as other types of stainless steel or composites or the like. Engaged with the side of the nozzle 24 opposite from the interior compartment 18 of the chamber 14 is a conduit-securing bolt 186. The conduit securing bolt 186 is threadably inserted into the bore 180 in the nozzle insert 192 to form a fluid passageway between the twenty-third conduit 74W of the fluid injection mechanism 20 and the chamber 182 of the nozzle 24. A seal, such as an O-ring, 188 is preferably positioned between the flange of the conduit securing bolt 186 and the exterior surface of the nozzle insert 192.

Referring to FIG. 1, the fluid injection mechanism 20 includes reservoirs 44A, 44B, or 44C for storing the fluid and conduits extending between the reservoirs 44A-44C and the chamber 14. Pumps 72A, 72B, or 72C remove fluid from the reservoirs 44A, 44B, or 44C and drive the fluid through the conduits toward the chamber 14. The first and second pumps 72A, 72B which are used with the rinse fluid 46 and the bio-burden removing fluid 48 are preferably liquid diaphragm pumps. The third pump 72C which is used with the sterilizing fluid 50 is preferably a modified liquid diaphragm pump. More specifically, the third pump 72C is preferably a liquid diaphragm pump that has been modified to also act as a metering pump. The modified third pump 72C permits improved control over the amount of sterilizing fluid 50 which is used by the apparatus 10.

Additionally, the fluid injection mechanism 20 includes air valves 76A, 76B, 76C, or 76D for supplying pressurized air to remove fluid from the conduits and propel the fluid toward the chamber 14. The fluid injection mechanism 20 further includes heaters 88A-88D, 90 to maintain the fluid at approximately the predetermined fluid temperature.

The first through fourth heaters are preferably part of an independent thermal control circuit. Each heater preferably, but not necessarily, comprises a heating element, such as copper or the like, which is wrapped around the heater chamber and sealed with a jacket that covers the heating element. A thermocouple is preferably combined with the heaters 88A-88B to allow for the detection of the temperature of the fluid contained therein. Each thermal control circuit monitors the temperature of the associated fluid and automatically powers the heater 88A-88D as necessary to bring the fluid substantially to the predetermined fluid temperature. Accordingly, each thermal control circuit preferably controls a respective heater so that all the controller 92 needs to monitor is the temperature of the fluid. Assuming the temperature of the fluid is within the predetermined range, the controller 92 will operate the rest of the liquid injection mechanism as further detailed below.

More specifically, the rinse fluid 46 is preferably contained within a first reservoir 44A, the bio-burden removal fluid 48 is preferably contained within a second reservoir 44B, and the sterilizing fluid 50 is preferably contained within a third reservoir 44C. Each of the reservoirs 44A-44C has an associated pump 72A-72C, which initially transports the fluid toward the chamber 14.

A heater is preferably not used to heat the bio-burden removing fluid 48 because the bio-burden removing fluid 48 is substantially brought to the predetermined fluid temperature due to the heat generated by the rinse fluid 46, the sterilizing fluid 50, the pressurized air, and the heater 90 which maintains the chamber 14 at the predetermined compartment temperature. Due to the relatively higher mass of the instrument 12 and the chamber 14, the bio-burden removing fluid 48 is heated to the predetermined fluid temperature without significantly altering the temperature of the instrument 12 or the chamber 14. Those of skill in the art will appreciate from this disclosure that a heater for the bio-burden removing fluid can be incorporated with the apparatus 10 without departing from the scope of the present invention.

Pressurized atmospheric air preferably enters the apparatus 10 via an inlet 94, which is attached to an air filter 96. The pressurized air is preferably supplied by a compressor (not shown) which is external to the apparatus 10. However, those of ordinary skill in the art will appreciate from this disclosure that a compressor could be incorporated with the apparatus 10 without departing from the scope of the present invention. The apparatus preferably uses about one cubic foot of air per minute at about seventy five pounds per square inch. However, those of ordinary skill in the art will appreciate from this disclosure that the amount of pressurized air that is used by the apparatus 10 can be modified depending on the size of the apparatus 10 and depending on the flow rates that the apparatus is designed to use without departing from the scope of the present invention.

The air filter 96 filters and guides the pressurized air to a pressure regulator 100, which is monitored via a pressure gauge 98. The pressure of the pressurized air is preferably in the range of between about 75 pounds per square inch and about 85 pounds per square inch. However, those of skill in the art will appreciate from this disclosure that the pressure of the pressurized air can be varied depending upon the specific components used to form the apparatus 10.

In the event of excessive pressure in the air filter 96, automatic discharge valves 102 open and cause air to be dumped from the apparatus via a seventh conduit 74G, through a fourteenth checkvalve 86N, and out through the apparatus outlet 66. Once the filtered air is transported past the pressure regulator 100, the pressurized air is heated using a first heater 88A and is then transported along a first conduit 74A. The pressure of the filtered air in the first conduit 74A is monitored by an inlet air pressure sensor 104. The inlet air pressure sensor 104 is preferably an electronic transducer. However, those of skill in the art will appreciate that any sensor capable of reliably monitoring the inlet air pressure can be used without departing from the scope of the present invention. The first conduit 74A supplies air to first through fourth air valves 76A-76D and a drain air valve 64 via second through sixth conduits 74B-74F respectively.

The checkvalves of the present invention are preferably acid resistant and relatively small sized. For example, the checkvalves of the present invention are preferably one half inch in length and one half inch in diameter. The checkvalves are preferably designed to interface with conduits that have an external diameter of about four millimeters.

Each of the first through fourth air valves 76A-76D and the drain air valve 64 are connected via an eighth conduit 74H to an air exhaust valve 188. Each of the air valves 76A-76D is shown in the first, or disengaged, position 40. While the first through fourth air valves 76A-76D, and the drain air valve 64 are in the first position 40, the exhaust valve 188 prevents pressurized air from remaining in the conduits connecting the respective air valves to the portion of the fluid injection mechanism 20 which transports the fluids, further detailed below. The first through fourth air valves 76A-76D and the drain air valve 64 are preferably SMC™ air valves. The air valves are compact and measure about a half inch in length and have a half inch diameter. Each air valve preferably has a power consumption of about one half a Watt.

Referring to the first air valve 76A, the first air valve 76A is biased into the first position 40 via a first input biasing element 78A. When the first air valve 76A is in the first position 40, any pressurized air in a ninth conduit 74I is diverted through the eighth conduit 74H to the exhaust valve 188. A switch 80A is capable of moving the first air valve 76A from the first position 40 into the second position 42 which causes the filtered pressurized air that is supplied via the second conduit 74B to be applied to the ninth conduit 74I and through a first checkvalve 86A.

Referring to the second air valve 76B, the second air valve 76B is biased into the first position 40 by a second input biasing element 78B. While the second air valve 76B is in the first position 40, any pressurized air in a tenth conduit 74J is diverted to the exhaust valve 188 via the eighth conduit 74H. A second switch 80B can move the second air valve 76B into the second position 42 which causes filtered, pressurized air in the third conduit 74C to be applied to the tenth conduit 74J and driven through a third checkvalve 86C into the eighteenth conduit 74R.

Referring to the third air valve 76C, the third air valve 76C is biased into the first position 40 by a third input biasing element 78C. While the third air valve 76C is in the first position 40, any pressurized air in an eleventh conduit 74K is diverted to the exhaust valve 188 via the eighth conduit 74H. A third switch 80C can move the third air valve 76C into the second position 42. When the third air valve 76C is in the second position 42, filtered pressurized air from the fourth conduit 74D is provided to the eleventh conduit 74K. When pressurized air is driven into the eleventh conduit 74K, the air is guided to an air diverter valve 108.

The air diverter valve 108 has a second diverter switch 110B capable of moving the air diverter valve 108 between a first position 40 and a second position 42. The air diverter valve 108 is shown in the second position 42 in FIG. 1. While the air diverter valve 108 is in the second position 42, air from the eleventh conduit 74K is provided to a nineteenth conduit 74S and driven through a ninth checkvalve 86I. When the air diverter valve 108 is in the first position 40, pressurized air from the eleventh conduit 74K is provided to a twentieth conduit 74T and driven through a sixth checkvalve 86F. The operation and positioning of the air diverter valve 108 is further discussed below.

Referring to the fourth air valve 76D, a fourth input biasing element 78D biases the fourth air valve 76D into the first position 40. While the fourth air valve 76D is in the first position 40, any pressurized air in twelfth conduit 74L is diverted to the exhaust valve 188 via the eighth conduit 74H. A fourth switch 80D is capable of moving the fourth air valve 76D into the second position 42. While the fourth air valve 76D is in the second position 42, filtered pressurized air from the fifth conduit 74E is provided to the twelfth conduit 74L and driven through an eleventh checkvalve 86K.

Referring to the drain air valve 64, the drain air valve 64 is biased into a first position 40 by a drain-biasing element 82. While the drain air valve 64 is in the first position 40, pressurized air in a thirteenth conduit 74M is diverted to the exhaust valve 188 via the eighth conduit 74H. A drain switch 84 is capable of moving the drain air valve 64 into the second position 42. While the drain air valve 64 is in the second position 42, pressurized air from a sixth conduit 74F is provided to the thirteenth conduit 74M and driven through a restrictor 190 and a thirteenth checkvalve 86M. The restrictor 190 reduces the flow of the filtered pressurized air through the thirteenth checkvalve 86M. The restrictor 190 is preferably used because the flow of the pressurized air from the drain air valve 64 is in excess of that which is desired to create a suction effect to remove fluid from the chamber, as further detailed below.

Each of the first through fourth switches 80A-80D and the drain switch 84 are preferably integral with the SMC™, or similar type, air valve and are air assisted switches. In other words the switches are moved partially using electric power and then, are moved the rest of the way using a portion of the pressurized air. However, those of ordinary skill in the art will appreciate from this disclosure that the first through fourth switches 80A-80D may be separate components from their respective air valves without departing from the scope of the present invention. For example, electrically operated solenoid switches that are controlled by the controller 92. However, those of ordinary skill in the art will appreciate from this disclosure that any type of switch used for the positioning of valves can be used without departing from the scope of the present invention.

Rinse fluid 46 is removed from the first reservoir 44A and driven through a fourteenth conduit 74N by the first pump 72A. During one complete sterilization operation of the apparatus 10 about fifty millimeters to about one hundred fifty milliliters of rinse fluid 46 is preferably used. However, those of ordinary skill in the art will appreciate from this disclosure that depending on the size of the apparatus 10 and depending upon the type of rinse fluid 46 used, the amount of rinse fluid 46 that is processed by the apparatus 10 during one complete sterilization operation can be varied without departing from the scope of the present invention. An exhaust valve 200 is attached to the first reservoir 44A to allow air to enter the first reservoir 44A and to reduce the amount of force that must be generated by the first pump 44A to remove the rinse fluid 46 from the first reservoir 44A. The rinse fluid 46 is then driven through a second checkvalve 86B to the second heater 88B. The second heater 88B ensures that the rinse fluid 46 is at the predetermined fluid temperature prior to the controller 92 applying the rinse fluid 46 to the instrument 12 contained within the chamber 14, further detailed below. To apply the rinse fluid 46 to the instrument 12 contained within the chamber 14, the first pump 72A in combination with the first, second, and fourth air valves 76A, 76B, and 76D drives the rinse fluid 46 into the chamber 14 as described below.

To transfer the rinse fluid 46 from the second heater 88B to the chamber 14, the first air valve 76A is moved into the second position 42 to provide pressurized air to the ninth conduit 74I. When pressurized air is transferred through the ninth conduit 74I, the pressurized air passes the first checkvalve 86A to push heated fluid from the second heater 88B into a seventeenth conduit 74Q which guides the rinse fluid 46 to the first fluid sensor 106A. Then, the rinse fluid 46 is driven the past the fourth checkvalve 86D and into the eighteenth conduit 74R.

The second air valve 76B is then moved into the second position 42 to transfer pressurized air into the tenth conduit 74J, past the third checkvalve 86C, and into the eighteenth conduit 74R to push the rinse fluid 46 toward a diverter valve 38. The diverter valve 38 guides the rinse fluid 46 (or either one of the bio-burden removing fluid 48 and the sterilizing fluid 50, as appropriate) toward either the portion 22 of the instrument 12 that is engaged by the chamber 14 or toward the fluid outlets 24 disposed in the walls of the chamber 14. When the diverter valve 38 is in the first position 40; the rinse fluid is transferred to the twenty-second conduit 74V and into the twenty-third conduit 74W. Then, the fourth air valve 76D is moved into the second position 42 to transfer pressurized air from the fifth conduit 74E to the twelfth conduit 74L and then through the eleventh checkvalve 86K. The pressurized air that is driven through the eleventh checkvalve 86K aids in driving the rinse fluid 46 contained in the twenty-third conduit 74W into the fluid outlets 24 for application onto the exterior 32 of the instrument 12 contained within the chamber 14.

Alternatively, when the diverter valve 38 is in the second position 42, the rinse fluid 46 is transferred to the twenty-sixth conduit 74Z which guides the rinse fluid 46 to the portion 22 of the instrument 12 that is engaged with the lid 16 of the chamber 14. A first diverter switch 110A enables the diverter valve 38 to send fluid to either the fluid outlets 24 or to the portion 22 of the instrument 12 that is engaged with the chamber 14. Thus, the rinse fluid 46 is transferred to the chamber 14 due to forces provided by the first pump 72A, the first air valve 76A, the second air valve 76B, and the fourth air valve 76D.

The first and second diverter switches 110A, 110B are preferably integral with their respective air valves and can be controlled by the controller 92. However, those of ordinary skill in the art will appreciate from this disclosure that the first and second diverter switches can be electrically operated solenoid switches, electric motors or the like.

To transfer the bio-burden removing fluid 48 from a second reservoir 44B to the chamber 14, a second pump 72B drives the bio-burden removing fluid 48 through a fifteenth conduit 74O past a second fluid sensor 106B and past a fifth checkvalve 86E. Then, the bio-burden removing fluid 48 enters the eighteenth conduit 74R and is guided toward the diverter valve 38. Then, second air valve 76B is moved into the second position to guide pressurized air from the third conduit 74C to the tenth conduit 74J to aid in driving the bio-burden removing fluid 48 through the eighteenth conduit 74R to the diverter valve 38. The apparatus 10 preferably uses between about six milliliters and about twelve milliliters of bio-burden removing fluid 48 during the complete sterilization process for one instrument 12. However, those of ordinary skill in the art will appreciate from this disclosure that depending on the size of the apparatus and the type of bio-burden removing fluid 48 used, that the amount of bio-burden removing fluid used can be varied without departing from the scope of the present invention.

Depending upon the position of the diverter valve 38, the bio-burden removing fluid 48 is directed toward either the portion 22 of the instrument 12 that is engaged by the chamber 14 or toward the nozzles 24 contained in the chamber 14. When the diverter valve 38 is in the first position 40, the bio-burden removing fluid 48 enters into the twenty-second conduit 74V and is guided to the twenty-third conduit 74W.

Then, the fourth air valve 76D is moved into the second position 42 causing pressurized air to move from the fifth conduit 74E to the twelfth conduit 74L to aid in driving the bio-burden removing fluid 48 from the twenty-third conduit 74W to the fluid outlets 24 in the chamber 14 for application of the bio-burden removing fluid 48 to the exterior 32 of the instrument 12.

When the diverter valve 38 is in the second position 42, the bio-burden removing fluid 48 is transferred to the twenty-sixth conduit 74Z which guides the bio-burden removing fluid 48 to the portion 22 of the instrument 12 which is engaged by the chamber 14. Thus, the bio-burden removing fluid 48 is transferred from the second reservoir 44B to the chamber 14 by the action of the second pump 72B, the second air valve 76B, and the fourth air valve 76D.

The sterilizing fluid 50 is transferred from the third reservoir 44C to the chamber 14 as follows. The third pump 72C transfers the sterilizing fluid 50 from the third reservoir 44C to a sixteenth conduit 74P and drives the sterilizing fluid 50 through a seventh checkvalve 86G. An exhaust valve 198 is attached to the third reservoir 44C to allow air to enter the third reservoir 44C and to reduce the amount of force that must be generated by the third pump 44C to remove the sterilizing fluid 50 from the third reservoir 44C. Then, the sterilizing fluid 50 is pumped into a third heater 86C, through an eighth checkvalve 86H, and into a fourth heater 88D. Once the Sterilizing solution has filled both the third and fourth heaters 88C, 88D, a third fluid sensor 106C indicates that a complete charge of the sterilizing fluid 50 is ready for application after being heated to the predetermined fluid temperature. The sterilizing fluid 50 is preferably applied two times during the sterilization of the instrument 12 (each time providing a full charge of sterilizing fluid 50 to the instrument). The second sterilizing fluid 50 treatment is preferably applied without an intervening rinse fluid 46 application to prevent as much dilution as possible. It is preferred that the total amount of sterilant used by the apparatus 10 during the sterilization of the instrument 12 be between about six milliliters and about thirty milliliters. However, those of ordinary skill in the art will appreciate from this disclosure that greater or lessor amounts of sterilant can be used without departing from the scope of the present invention.

Then, once the sterilizing fluid 50 that is in the twenty-first conduit 74U (i.e.: in the third and fourth heaters 88C, 88D) has reached the predetermined fluid temperature, the third air valve 76C is moved into the second position 42 causing pressurized air to enter the eleventh conduit 74K. The pressurized air is guided to the air diverter valve 108, which is switched into the second position 42 to guide air into the nineteenth conduit 74S, and through the ninth checkvalve 86I. This causes the pressurized air to drive the sterilizing fluid 50 which is contained above the eighth checkvalve 86H through a tenth checkvalve 86J and into the diverter valve 38. Depending upon the position of the diverter valve 38, the sterilizing fluid 50 is either guided toward the fluid outlets 24 in the chamber 14 or toward the portion 22 of the instrument 12 which is engaged by the chamber 14.

When the diverter valve 38 is in the first position 40, the sterilizing fluid 50 is transferred to the twenty-second conduit 74V and into the twenty-third conduit 74W. Then, the fourth air valve 76D is moved into the second position 42 causing pressurized air to enter the twelfth conduit 74L. This causes pressurized air to pass through the eleventh checkvalve 86K and to drive the sterilizing fluid 50 through the twenty-third conduit 74W into the fluid outlets 24 for application to the exterior 32 of the instrument 12 contained within the chamber 14. Alternatively, when the diverter valve 38 is in the second position 42, the sterilizing fluid 50 is transferred to the twenty-sixth conduit 74Z which guides the sterilizing fluid 50 to the portion 22 of the instrument 12 which is engaged by the lid 16 of the chamber 14.

After the application of the sterilizing fluid 50 which was temporarily positioned above the eighth checkvalve 86H is completed, the air diverter valve 108 is moved into the first position 40 causing pressurized air to enter the twentieth conduit 74T and to pass through the sixth checkvalve 86F. This results in the pressurized air driving the remaining sterilizing fluid 50 that is present on the right side of the seventh checkvalve 86G toward the diverter valve 38. Once the remaining sterilizing fluid 50 reaches the diverter valve 38, the sterilizing fluid 50 is guided toward either the fluid outlets 24 in the chamber 14 or toward the portion 22 of the instrument 12 which is engaged with the lid 16 of the chamber 14, as described above.

The chamber 14 further includes at least another fluid outlet 26 to direct the flow of the fluid onto the portion 22 of the instrument 12 engaged by the chamber 14. Accordingly, as described above, fluid is guided through the twenty-sixth conduit 74Z, the fluid is directed towards the portion 22 of the instrument 12 by the other fluid outlet 26. When the instrument 12 has an interior 28 that has a fluid pathway connection to the portion 22 of the instrument 12 engaged by the chamber 14, the other fluid outlet 26 also directs a flow of the fluid into an interior 28 of the instrument 12. As the apparatus 10 of the present invention is preferably used with dental handpieces, the interior 28 of the instrument 12 is sterilized by the application of the sterilizing fluid 50 to the inside of the lumens 124A, 124B. However, those of skill in the art will appreciate from the present invention that the sterilizing apparatus 10 may be used with an instrument 12 not having an interior 28 without departing from the scope of the present invention.

The fluid injection mechanism 20 alternatingly supplies a flow of the fluid to either the one fluid outlet 24 or into the other fluid outlet 26. As detailed above, the fluid injection mechanism 20 includes a diverter valve 38 for alternately supplying a flow of the fluid to the one fluid outlet 24 and to the other fluid outlet 26. While the preferred embodiment of the present invention preferably alternately directs a flow of fluid to either the portion 22 of the instrument 12 engaged by the chamber 14 or to the nozzles 24 of the chamber 14, those of skill in the art will appreciate from this disclosure that the fluid can be supplied simultaneously to both the nozzles 24 and to the portion 22 of the instrument 12 that is engaged by the chamber 14 without departing from the scope of the present invention. For example, the diverter valve 38 can be replaced by a flow divider (not shown) or the like, to simultaneously apply the fluid to both the exterior 32 of the instrument 12 and to the portion 22 of the instrument 12 that is engaged by the chamber 14.

Referring to FIG. 1, the apparatus 10 further includes a drain 52 for removing fluid from the chamber 14, and a drain valve 54 for opening and closing the drain 52. The drain valve 54 is biased into a closed position 56 by a drain valve-biasing element 60. When the drain valve 54 is in the closed position, the fluid is prevented from exiting the chamber 14 by a twelfth checkvalve 86L. A drain switch 68 is capable of moving the drain valve 54 into an open position 58 which allows the fluid to drain from the chamber 14 into a twenty-fourth conduit 74X, which forms a waste line 70.

To facilitate draining fluid from the chamber 14, the drain air valve 64 creates a vacuum to pull the fluid out of the twenty-fourth conduit 74X. More specifically, the drain air valve 64 is shown in FIG. 1 in the first position 40. The drain air valve 64 is biased into the first position 40 by a drain air valve biasing element 82. While the drain air valve 64 is in the first position 40, pressurized air in the thirteenth conduit 74M is transported to the exhaust valve 188 via the eighth conduit 74H.

A drain switch 84 is capable of moving the drain air valve 64 into the second position 42 which allows the drain air valve 64 to supply pressurized air to drive the fluid along the waste line 70 and through a drain nozzle 62, which is attached along the waste line 70. When the drain air valve 64 is in the second position, pressurized air from the sixth conduit 74F is provided to the thirteenth conduit 74M, through the restrictor 190, through the thirteenth checkvalve 86M, and into the waste line nozzle 62.

The airflow through the thirteenth conduit 74M creates a suction effect that pulls the fluid from the twenty-fourth conduit 74X and drives the fluid into a twenty-fifth conduit 74Y. Then, by opening the automatic discharge valves 102 a predetermined amount, air is propelled through the seventh conduit 74G and past the fourteenth checkvalve 86N. The flow of air through the seventh conduit 74G creates a further suction effect to pull the fluid from the twenty-fifth conduit 74Y to a waste line outlet 66 through which the fluid is expelled from the apparatus 10.

A method of sterilizing the instrument 12, which has an exterior surface 32 at substantially room temperature, preferably involves attaching the instrument 12 to the instrument holder 300. The method of the present invention preferably, but not necessarily, also includes the steps of determining via a first sensor 36A whether the chamber 14 is closed and determining via a second sensor 36B whether an instrument 12 is enclosed in the chamber 14 prior to beginning the removing of bio-burden.

Afterwards, the instrument is preferably secured inside of the chamber 14 by removably engaging a portion 22 of the instrument 12 to the chamber 14. After an instrument 12 is placed within the chamber 14, bio-burden is removed from the instrument 12 by exposing the instrument 12 to at least one bio-burden removing fluid 48 while maintaining the chamber 14 and the at least one bio-burden removing fluid 48 at about a first predetermined temperature.

While exposing the instrument 12 to at least one bio-burden removing fluid 48, the instrument holder 300 alternately uses the first and second cams 374A, 374B and the third and fourth 378A, 378B to hold the instrument 12. By alternately using pairs of cams that are offset along the outer surface of the instrument 12, the entire exterior surface of the instrument 12 is exposed to the at least one bio-burden removing fluid.

The removing of bio-burden includes using a protease fluid to wash the exterior surface 32 of the instrument 12 and to wash the portion 22 of the instrument 12 secured to the chamber 14. When using the sterilizer 10 with a dental handpiece, or another instrument 12 having an interior which has a fluid pathway connection to the portion 22 of the instrument 12 that is engaged with the chamber 14, the step of removing bio-burden preferably includes using the protease fluid to wash an interior 28 of the instrument 12 and to wash the portion of the instrument 12 secured to the chamber 14. Referring to FIG. 6, lumen engaging conduits 342 are preferably used to transfer the protease fluid to the lumens of the dental handpiece 12.

Additionally, when using a dental handpiece, or other instrument 12 having an interior connected by a fluid pathway to the portion 22 of the instrument 12 engaged by the chamber, it is preferred, but not necessary, that the step of removing bio-burden includes using a rinse fluid 46 to wash an interior surface 28 of the instrument 12. When using the rinse fluid, it is preferable to alternately use the first and second cams 374A, 374B and the third and fourth cams 378A, 378B to allow the rinse fluid to contact the entire exterior surface of the instrument 12.

The step of removing bio-burden preferably includes using the rinse fluid 46 after using the protease fluid to rinse the exterior surface 32 of the instrument 12 and to rinse the portion 22 of the instrument 12 secured to the chamber 14. Furthermore, the step of removing bio-burden includes alternately driving the at least one bio-burden removing fluid 48 against the exterior surface 32 of the instrument 12 and against a portion 22 of the instrument 12 engaged by the chamber 14.

The method also includes the step of sterilizing the instrument 12, including the portion 22 of the instrument 12 engaged by the chamber 14, by exposing the instrument 12 to at least one sterilizing fluid 50 while maintaining the chamber 14 and the at least one sterilizing fluid 50 at about a second predetermined temperature. While exposing the instrument 12 to at least one sterilizing fluid 50, the first and second cams 374A, 374B alternate with the third and fourth cams 378A, 378B to secure the instrument 12 and thus, allow the sterilizing fluid 50 to contact the entire exterior surface 32 of the instrument 12. Preferably, the first predetermined temperature and the second predetermined temperature are within the range of between about fifty-five degrees Fahrenheit and about ninety-five degrees Fahrenheit. More preferably, the first predetermined temperature and the second predetermined temperature are maintained within the range of about ninety degrees Fahrenheit and about ninety-four degrees Fahrenheit. The sterilizing of the instrument 12 preferably includes using the at least one sterilizing fluid 50 which comprises a peracetic acid to sterilize the exterior surface 32 of the instrument 12 and to sterilize the portion 22 of the instrument 12 secured to the chamber 14. When using the method of the present invention with a dental handpiece, or other instrument 12 having an interior having a fluid pathway to the portion 22, the step of sterilizing the instrument 12 preferably includes using the at least one sterilizing fluid 50 to sterilize an interior 28 of the instrument 12.

Additionally, the step of sterilizing the instrument 12 includes exposing the instrument 12 to the at least one sterilizing fluid 50 for a predetermined period of time. The predetermined period time is preferably between about three minutes and about five minutes. While it is preferred that the predetermined period of time be between about three minutes and about five minutes, those of skill in the art will appreciate from this disclosure that the period of time can be varied depending upon the particular sterilizing fluid 50 being used by the apparatus 10 to sterilize the instrument 12. Additionally, the predetermined period of time can also vary depending upon the specific concentration of the sterilizing fluid 50 which is used.

The method of sterilizing the instrument 12 preferably includes the following steps: applying the at least one sterilizing fluid 50 which comprises a peracetic acid to sterilize the exterior surface 32 of the instrument 12 and to sterilize the portion 22 of the instrument secured to the chamber; exposing the instrument 12 to the at least one sterilizing fluid 50 for a predetermined period of time; removing the at least one sterilizing fluid 50 from the chamber 14; and repeating the above steps of applying the fluid and then exposing the instrument 12.

Additionally, it is preferable to drain the at least one sterilizing fluid 50 from the chamber 14 after the step of sterilizing the instrument 12. The step of sterilizing the instrument 12 preferably includes alternately driving the at least one sterilizing fluid 50 against the exterior surface 32 of the instrument 12 and against the portion 22 of the instrument 12 engaged by the chamber 14.

In operation, the apparatus for sterilizing an instrument is preferably used as follows. Referring the FIGS. 6 and 6, a portion 22 of the instrument 12 is inserted into the instrument holder 300.

Referring to FIG. 6, with the instrument 12 secured to the base 302 of the instrument holder 300, the combination of the instrument holder 300 and the instrument 12 are inserted into the chamber 14. Referring to FIG. 1, once the instrument holder 300 is secured to the chamber 14, a first sensor 36A sends a lid-closed-signal to the controller 92. Then, a second sensor 36B determines whether an instrument 12 is positioned within the chamber 14. If the second sensor 36B detects an instrument 12 within the chamber 14, an instrument-presence-signal is sent to a controller 92. A third sensor 36C is then used to detect a compartment temperature and sends a temperature-signal to the controller 92. If the interior compartment 18 is not generally at the predetermined compartment temperature, then the controller 92 adjusts the heater 90 which surrounds the chamber 14 until the interior compartment 18 is generally at the predetermined compartment temperature.

Next, the controller 92 activates the first pump 72A to drive the rinse fluid 46 from the first reservoir 44A through the fourteenth conduit 74N, through the second checkvalve 86B, and into the second heater 88B until the first fluid detector 106A detects that the appropriate amount of the rinse fluid 46 is present in the seventeenth conduit 74Q.

Then, the first pump 72A preferably terminates pumping the rinse fluid 46 while the second heater 88B, if necessary, brings the rinse fluid 46 up to the predetermined fluid temperature. Once the rinse fluid 46 is generally at the predetermined fluid temperature, the controller 92 causes the first input valve switch 80A to move the first air valve 76A into the second position 42 causing pressurized air to flow from the second conduit 74B to the ninth conduit 74I, through the first checkvalve 86A, and to push the charge of rinse fluid 46 in the second heater 88B past the fourth checkvalve 86D and into the eighteenth conduit 74R.

Then, the controller 92 causes the second input valve switch 80B to move the second air valve 76B into the second position 42 causing pressurized air to flow from the third conduit 74C into the tenth conduit 74J, through the third checkvalve 86C, and to combine with the pressurized air supplied by the first air valve 76A to push the rinse fluid 46 through the diverter valve 38 and into the chamber 14. As detailed above, the diverter valve 38 alternatingly sends a flow of the rinse fluid 46 to either the nozzles 24 in the chamber 14 or the other outlet 26 in the lid 16 of the chamber 14. As detailed above, when the diverter valve 38 sends fluid to the nozzles 24, the fourth air valve 76D is also used to supply pressurized air.

The first application of the rinse fluid 46 to the instrument 12 serves as a pre-wash for the instrument 12. While the rinse fluid 46 is being applied to the handpiece 12, the drain 52 is preferably closed by the drain valve 54. Once the application of the rinse fluid 46 to the exterior of the instrument 12 and to the portion 22 of the instrument 12 engaged by the chamber 14 (and to any interior 28 of the handpiece 12 which is connected by a fluid pathway connection to the portion 22 of the handpiece 12 engaged by the chamber 14) is complete, the drain 52 is opened.

To open the drain 52, the controller 92 activates the drain switch 68 which moves the drain valve 54 into the open position 58 while generally simultaneously activating the drain air valve 64. When the controller 92 activates the drain air valve 64, the drain air valve switch 84 moves the drain air valve 64 into the second position 42 causing pressurized air to flow from the sixth conduit 74F into the thirteenth conduit 74M, through the restrictor 190, through the thirteenth checkvalve 86M, and into the twenty-fifth conduit 74Y.

The flow of the pressurized air from the thirteenth conduit 74M into the twenty-fifth conduit 74Y creates a suction affect causing the rinse fluid 46 which is present in the chamber 14 to be drawn into the twenty-fourth conduit 74X. Then, the controller 92 causes the automatic discharge 102 to open and sends pressurized air into the seventh conduit 74G, through the fourteenth checkvalve 86N, and into the twenty-fifth conduit 74Y. The flow of pressurized air from the seventh conduit 74G into the twenty-fifth conduit 74Y creates a vacuum affect which further draws the remaining rinse fluid 46 from the chamber 14 and through the waste line outlet 66.

While the rinse fluid 46 is being removed from the chamber 14, a partial drying phase is preferably, but not necessarily, initiated. The controller 92 moves the second and fourth air valves 76B, 76D into the second position 42 causing pressurized air to be guided toward the fluid outlets 24 in the chamber 14 sides and to the other fluid outlet 26. The application of pressurized air to the instrument 12 does not “dry” the instrument 12, but the pressurized air does remove the larger water droplets from the instrument 12.

Once the rinse fluid 46 has been removed from the chamber 14 and the partial drying phase is complete, the controller 92 deactivates the drain switch 68 causing the drain biasing element 60 to move the drain valve 54 into the closed position 56. It is then preferable that the controller 92 deactivates the first air valve 76A, the second air valve 76B, the drain air valve 64, and the automatic discharge 102 in preparation for treating the instrument 12 with the next fluid as detailed below. However, those of skill in the art will appreciate from this disclosure that the above-mentioned valves and the auto-discharge 102 can be closed at another point in the process without departing from the scope of the present invention as long as the proper transfer of the rinse fluid 46 is not interrupted.

After the above-described application of the rinse fluid 46 to the instrument 12, the apparatus uses the bio-burden removing fluid 48 to clean the instrument 12 as follows. The second pump 72B drives the bio-burden removing fluid 48 into the fifteenth conduit 74O until the second fluid sensor 106B determines that the appropriate amount of bio-burden removing fluid 48 is present in the fifteenth conduit 74O. Then, the bio-burden removing fluid is pumped past the fifth checkvalve 86E and into the eighteenth conduit 74R. The controller 92 then activates the second air valve 76B by causing the second input valve switch 80B to move the second air valve 76B into the second position 42. This causes pressurized air to be guided from the third conduit 74C into the tenth conduit 74J, to be guided past the third checkvalve 86C, and to drive the bio-burden removing fluid 48 from the eighteenth conduit 74R to the diverter valve 38.

As described above, the diverter valve 38 causes the bio-burden removing fluid 48 to be guided to either the nozzles 24 in the chamber 14 or to the instrument holder 300 which secures the instrument 12 to the chamber 14. After the bio-burden removing fluid 48 has been applied to the instrument 12, the fluid is preferably left in contact with the instrument 12 for about one minute to about ten minutes (more preferably for about one minute to about five minutes). Then, a second application of the bio-burden removing fluid 48 is preferably applied to the instrument 12 and left in contact with the instrument as detailed above. Thus, the total contact time of the bio-burden removing fluid 48 with the instrument 12 for two applications of the bio-burden removing fluid 48 is preferably, but not necessarily, about two minutes to about 20 minutes (more preferably for about two minutes to about ten minutes).

After the second application of the bio-burden removing fluid 48 has been in contact with the handpiece 12 for the preferred period of time, the controller 92 activates the drain switch 68 to cause the drain valve 54 to move into the open position 58. Then, the controller 92 uses the drain air valve 64 and the auto-discharge 102 to remove the bio-burden removing fluid 48 from the chamber 14 in the manner described above.

After the completion of the treating of the instrument 12 with the bio-burden removing fluid 48, it is preferable to use the rinse fluid 46 to remove any remaining bio-burden removing fluid 48 from the instrument 12. Thus, the controller 92 causes the apparatus 10 to again apply the rinse fluid 46 to the instrument 12 in the manner described above. After the rinse fluid 46 has been applied to the instrument, a second partial dry phase is preferably performed in the same manner as detailed above.

Once the bio-burden has been removed from the instrument 12 and the partial dry phase has been completed, the pathogens, which are present on the instrument 12, are exposed to facilitate a total kill of the pathogens using the sterilizing fluid 50. To apply the sterilizing fluid 50 to the handpiece 12, the apparatus 10 operates as follows. The controller 92 activates the third pump 72C to drive the sterilizing fluid 50 into the sixteenth conduit 74P, through the seventh checkvalve 86G, and into the twenty-first conduit 74U until the third and fourth heaters 88C, 88D are filled with the sterilizing fluid 50 and the third fluid sensor 106C indicates a full charge of the sterilizing fluid 50 is present in the twenty-first conduit 74U.

Then, the controller 92 activates the third input valve switch 80C to move the third air valve 76C into the second position 42 causing pressurized air to flow from the fourth conduit 74D into the eleventh conduit 74K. At approximately the same time, the controller 92 uses the second diverter switch 110B to move the air diverter valve 108 into the second position 42 causing the pressurized air in the eleventh conduit 74K to push the sterilizing fluid 50 that is positioned above the eighth checkvalve 86H to the diverter valve 38. Then, the diverter valve 38 guides the sterilizing fluid 50 toward the chamber 14 as described above.

Once the sterilizing fluid 50 that is positioned above the eighth checkvalve 86H has been applied to the instrument 12, the controller 92 uses the second diverter switch 110B to move the air diverter valve 108 into the first position 40 causing the pressurized air in the eleventh conduit 74K to drive the remaining sterilizing fluid 50 which is present in the twenty-first conduit 74U to the diverter valve 38. Then, the remaining sterilizing fluid 50 is applied to the handpiece 12.

Once the sterilizing fluid 50 has been applied to the handpiece 12, it is preferable that the drain 52 remain closed and that the instrument 12 remain exposed to the sterilizing fluid for a predetermined period of time. As discussed above, it is preferable that the instrument 12 be exposed to the sterilizing fluid 50 for a period of time between about three minutes and about seven minutes (more preferably between about three minutes and about four and one half minutes). Once the instrument 12 has been exposed to the sterilizing fluid 50 for the predetermined period of time, the sterilizing fluid 50 is removed from the chamber 14 in a manner similar to that described above with reference to the rinse fluid 46. Once the application of the sterilizing fluid 50 is complete, it is preferable to again expose the instrument 12 to a second application of the sterilizing fluid 50. Once the second application of the sterilizing fluid 50 is complete and the instrument 12 has been left in contact with the second application of the sterilizing fluid 50 for a predetermined period of time, the instrument 12 is again rinsed using the rinse fluid 46. After the final application of the rinse fluid 46, another partial drying phase is preferably performed and the instrument 12 sterilization process is complete.

It is recognized by those skilled in the art, that changes may be made to the above-described embodiment of the present invention without departing from the broad inventive concept thereof. It is understood, therefor, that this invention is not limited to the particular embodiment disclosed, but is intended to cover all modifications which are within the spirit and scope of the invention.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An instrument holder for securing an instrument in a chamber of a sterilizer, the instrument having an external surface and a proximal end, the sterilizer having a source of fluid, the instrument holder comprising:
a housing having a base with a bore therethrough;
an instrument coupler for engaging the proximal end of the instrument, the instrument coupler movable within the bore; and
an instrument retention assembly connected to the base, the instrument retention assembly comprising a first grip and a second grip for alternately securing the instrument to the base.

2. The instrument holder according to claim 1, wherein the instrument coupler comprises:
an instrument engaging end engagable with the proximal end of the instrument;
an intake port for receiving fluid from the source of fluid;
a passageway in fluid communication with the intake port; and
a discharge port for discharging fluid to the exterior surface of the instrument, the discharge port in fluid communication with the passageway when the proximal end of the instrument is engaged with the instrument engaging end of the instrument coupler.

3. The instrument holder according to claim 2, wherein the passageway comprises a plurality of lumen engaging conduits, and the instrument further comprises a manifold in fluid communication with the intake port and at least one conduit of the plurality of lumen engaging conduits.

4. The instrument holder according to claim 3, wherein the instrument is a dental handpiece having at least a first lumen for injecting fluid into the handpiece and a second lumen for discharging fluid from the handpiece, each lumen projecting from the proximal end of the instrument, and the plurality of lumen engaging conduits includes a first lumen engaging conduit and a second lumen engaging conduit, the second lumen engaging conduit in fluid communication with the discharge port, the first and second lumen engaging conduits positioned for engagement with the first and second lumens, respectively, when the instrument engaging end of the instrument coupler is engaged with the proximal end of the handpiece.

5. The instrument holder according to claim 1, wherein the instrument coupler has a position biasing member for maintaining a predetermined contact force between the instrument engaging end of the instrument coupler and the proximal end of the instrument, the position biasing member having a first end connected to the housing and a second end connected to the instrument coupler.

6. The instrument holder according to claim 1, wherein the instrument coupler further comprises an annular seal in slideable frictional engagement with the bore.

7. The instrument holder according to claim 1, wherein the instrument coupler comprises:
an instrument engaging end engagable with the proximal end of the instrument;
a position biasing member for maintaining a predetermined contact force between the instrument engaging end of the instrument coupler and the proximal end of the instrument, the position biasing member having a first end connected to the housing and a second end connected to the instrument coupler;
an annular seal in slideable frictional engagement with the bore;
an intake port for receiving fluid from the source of fluid;
a manifold in fluid communication with the intake port;
a passageway comprising a plurality of lumen engaging conduits, at least one conduit of the plurality of lumen engaging conduits in fluid communication with the manifold; and
a discharge port for discharging fluid to the exterior surface of the instrument, the discharge port in fluid communication with the passageway when the proximal end of the instrument is engaged with the instrument engaging end of the instrument coupler.

8. The instrument holder according to claim 7, wherein the instrument is a dental handpiece having at least a first lumen for injecting fluid into the handpiece and a second lumen for discharging fluid from the handpiece, each lumen projecting from the proximal end of the instrument, and the plurality of lumen engaging conduits includes a first lumen engaging conduit and a second lumen engaging conduit, the second lumen engaging conduit in fluid communication with the discharge port, the first and second lumen engaging conduits positioned for engagement with the first and second lumens, respectively, when the instrument engaging end of the instrument coupler is engaged with the proximal end of the handpiece.

9. The instrument holder according to claim 1, wherein the first grip comprises a first pair of opposing cams and the second grip comprises a second pair of opposing cams, each cam of the first and second grips pivotably connected to the base and pivotable between a disengaged position and an engaged position.

10. The instrument holder according to claim 9, wherein the first pair of cams comprises a first cam and a second cam, the first and second cams driven by a first actuator and a second actuator, respectively, the first and second actuators synchronized for common actuation, and the second pair of cams comprises a third cam and a fourth cam, the third and fourth cams driven by a third actuator and a fourth actuator, respectively, the third and fourth actuators synchronized for common actuation.

11. The instrument holder according to claim 10, wherein the first, second, third, and fourth actuators are configured to bias the first, second, third, and fourth cams, respectively, in the disengaged position.

12. The instrument holder according to claim 11, wherein the first, second, third, and fourth actuators are pneumatic actuators.

13. The instrument holder according to claim 9, wherein the first grip and the second grip form an arrangement operable in a plurality of configurations comprising:
a disengaged configuration, wherein each cam of the first and second grips is in the disengaged position and the proximal end of the instrument is removably engagable with the instrument coupler;
a first configuration, wherein the first pair of opposing cams of the first grip is in the engaged position and secures the instrument to the base, and the exterior surface of the instrument along opposed first radial arcs is exposed;
a second configuration, wherein the second pair of opposing cams of the second grip is in the engaged position and secures the instrument to the base, and the exterior surface of the instrument along opposed second radial arcs is exposed; and
a third configuration, wherein the first pair of cams and the second pair of cams are in the engaged position and secure the instrument to the base, and the exterior surface of the instrument along a plurality of third radial arcs is exposed.

14. The instrument according to claim 1, wherein the retention assembly further comprises a retainer ring between the first grip and the instrument and between the second grip and the instrument.

15. The instrument holder according to claim 14, wherein the first grip, the second grip, and the retainer ring form an arrangement operable in a plurality of configurations comprising:
a disengaged configuration, wherein the first and second grips are spaced from the retainer ring and the retainer ring is spaced from the instrument;
a first configuration, wherein the retainer ring is elastically deformed by the first grip and secures the instrument to the base, and the exterior surface of the instrument along opposed first radial arcs is exposed;
a second configuration, wherein the retainer ring is elastically deformed by the second grip and secures the instrument to the base, and the exterior surface of the instrument along opposed second radial arcs is exposed; and
a third configuration, wherein the retainer ring is elastically deformed by the first grip and the second grip and secures the instrument to the base, and the exterior surface of the instrument along a plurality of third radial arcs is exposed.

16. The instrument holder according to claim 1, wherein the first grip is a first ring and the second grip is a second ring, the first and second rings spaced apart and each ring having a radially inwardly facing surface that is pneumatically expandable.

17. An instrument holder for securing an instrument in a chamber of a sterilizer, the instrument having an external surface and a proximal end, the sterilizer having a source of fluid, the instrument holder comprising:
a housing having a base with a bore therethrough;
an instrument coupler for engaging the proximal end of the instrument, the instrument coupler movable within the bore, the instrument coupler comprising:
an instrument engaging end engagable with the proximal end of the instrument;
an intake port for receiving fluid from the source of fluid;
a passageway in fluid communication with the intake port; and
a discharge port for discharging fluid to the exterior surface of the instrument, the discharge port in fluid communication with the passageway when the proximal end of the instrument is engaged with the instrument engaging end of the instrument coupler; and
an instrument retention assembly connected to the base, the instrument retention assembly comprising:
a first grip comprising a first pair of opposing cams and a second grip comprising a second pair of opposing cams, each cam pivotably connected to the base and pivotable between a disengaged position and an engaged position.

18. The instrument holder according to claim 17, wherein
the passageway comprises a plurality of lumen engaging conduits;
the instrument coupler further comprises a manifold in fluid communication with the intake port and at least one conduit of the plurality of lumen engaging conduits;
the first pair of opposing cams comprises a first cam and a second cam, the first and second cams driven by a first actuator and a second actuator, respectively, the first and second actuators synchronized for common actuation; and
the second pair of opposing cams comprises a third cam and a fourth cam, the third and fourth cams driven by a third actuator and a fourth actuator, respectively, the third and fourth actuators synchronized for common actuation.

19. An instrument holder for securing an instrument in a chamber of a sterilizer, the instrument having an external surface, a proximal end and a plurality of lumens, the chamber has a sealable opening for receiving the instrument holder, and the sterilizer has a source of fluid, the instrument holder comprising:
a housing having a base with a bore therethrough;
an instrument coupler for engaging the proximal end of the instrument, the instrument coupler movable within the bore, the instrument coupler comprising:
an instrument engaging end engagable with the proximal end of the instrument;
a position biasing member for maintaining a predetermined contact force between the instrument engaging end of the instrument coupler and the proximal end of the instrument, the position biasing member having a first end connected to the housing and a second end connected to the instrument coupler;
an annular seal in slideable frictional engagement with the bore;
an intake port for receiving fluid from the source of fluid;
a manifold in fluid communication with the intake port;
a passageway comprising a plurality of lumen engaging conduits, at least one conduit of the plurality of lumen engaging conduits in fluid communication with the manifold; and
a discharge port for discharging fluid to the exterior surface of the instrument, the discharge port in fluid communication with the passageway when the proximal end of the instrument is engaged with the instrument engaging end of the instrument coupler; and
an instrument retention assembly connected to the base, the instrument retention assembly comprising:
a first grip comprising a first pair of opposing cams comprising a first cam and a second cam, the first and second cams driven by a first actuator and a second actuator, respectively, the first and second actuators synchronized for common actuation;
a second grip comprising a second pair of opposing cams comprising a third cam and a fourth cam, the third and fourth cams driven by a third actuator and a fourth actuator, respectively, the third and fourth actuators synchronized for common actuation,
wherein the first, second, third, and fourth cams are pivotably connected to the base and pivotable between a disengaged position and an engaged position, and
wherein the first grip and the second grip form an arrangement operable in a plurality of coinfigurations comprising:
a disengaged configuration, wherein each cam of the first and second grips is in the disengaged position and the proximal end of the instrument is removably engagable with the instrument coupler;
a first configuration, wherein the first pair of opposing cams of the first grip are in the engaged position and secure the instrument to the base, and the exterior surface of the instrument along opposed first radial arcs is exposed;
a second configuration, wherein the second pair of opposing cams of the second grip are in the engaged position and secure the instrument to the base, and the exterior surface of the instrument along opposed second radial arcs is exposed; and
a third configuration, wherein the first pair of cams and the second pair of cams are in the engaged position and secure the instrument to the base, and the exterior surface of the instrument along a plurality of third radial arcs is exposed.

20. The instrument holding device according to claim 19, wherein the instrument is a dental handpiece having at least a first lumen for injecting fluid into the handpiece and a second lumen for discharging fluid from the handpiece, each lumen projecting from the proximal end of the instrument, and the plurality of lumen engaging conduits includes a first lumen engaging conduit and a second lumen engaging conduit, the second lumen engaging conduit in fluid communication with the discharge port, the first and second lumen engaging conduits positioned for engagement with the first and second lumens, respectively, when the instrument engaging end of the instrument coupler is engaged with the proximal end of the handpiece.
